(19) United States
(12) Patent Application Publication
(10) Pub. No.: US 2025/0186458 A1
(43) Pub. Date: Jun. 12, 2025

(54) METHODS FOR THE TREATMENT OF MILD COGNITIVE IMPAIRMENT

(71) Applicant: BIOVIE INC., Carson City, NV (US)

(72) Inventors: Clarence Nathaniel Ahlem, San Diego, CA (US); Christopher L. Reading, San Diego, CA (US); Joseph M. Palumbo, Philadelphia, PA (US); Jeffrey Zhang, San Francisco, CA (US)

(21) Appl. No.: 19/055,380

(22) Filed: Feb. 17, 2025

Related U.S. Application Data
(63) Continuation of application No. PCT/US2023/032004, filed on Sep. 5, 2023.
(60) Provisional application No. 63/498,703, filed on Apr. 27, 2023.
(60) Provisional application No. 63/374,631, filed on Sep. 6, 2022.

(51) Int. Cl. A61K 31/568; A61K 9/00; A61K 9/48; A61P 25/28

(52) U.S. Cl. CPC A61K 31/568; A61K 9/0053; A61P 25/28; A61K 9/48

(57) ABSTRACT

Disclosed herein are compositions and methods useful for the treatment or amelioration of various diseases, disorders, or conditions. Some aspects pertain to a pharmaceutical composition comprising 17-ethynyl-10R, 13S-dimethyl 2, 3, 4, 7, 8R, 9S, 10, 11, 12, 13, 14S, 15, 16, 17-hexadecahydro-1H-cyclopenta[a]phenanthrene-3R, 7R, 17S-triol, including solid states thereof. Also presented herein is the surprising discovery that exposing a subject to the compositions disclosed herein can treat, reduce, or ameliorate symptoms of a neurodegenerative condition.

### CROSS REFERENCE TO RELATED APPLICATIONS

This application is a continuation of PCT App. No. PCT/US2023/032004, filed on Sep. 5, 2023, which claims the benefit of priority to U.S. Provisional Application No. 63/498,703, filed Apr. 27, 2023, and U.S. Provisional Application No. 63/374,631 filed Sep. 6, 2022. All of the foregoing applications are incorporated herein by reference in their entireties for all purposes.

### FIELD OF THE DISCLOSURE

The present disclosure relates to the field of chemistry and medicine. More particularly, the present disclosure relates generally to methods for the treatment of conditions related to mild cognitive impairment using 17-ethynyl-10R, 13S-dimethyl 2, 3, 4, 7, 8R, 9S, 10, 11, 12, 13, 14S, 15, 16, 17-hexadecahydro-1H-cyclopenta[a]phenanthrene-3R, 7R, 17S-triol.

### BACKGROUND

Mild cognitive impairment (MCI) and dementia result in substantial cost to society in terms of financial and humanistic burden (Connolly 2020), which will only increase over time. Alzheimer's dementia (AD) and related degenerative conditions that result in MCI tend to be characterized by abnormal deposits of protein in the brain, such as amyloid (which forms plaques between neurons) and phospho-tau (which creates neurofibrillary tangles inside neurons). These protein depositions disrupt neuronal function and connectivity. Progression of conditions of the AD type are prompted by neuroinflammation, which may drive insulin desensitization. The insulin receptor is responsible for activating transfer enzymes (or kinases) which in turn promote cell growth and synaptic activity. Inflammation, such as via tumor necrosis factor (TNF), can block such signaling and result in neuronal damage and eventual neurodegeneration.

Currently, an estimated 5.7 million Americans have Alzheimer's disease and by 2050 it is projected that 13.8 million Americans will have it. Alzheimer's disease is the sixth leading cause of death in the United States. It is estimated that the total cost for everyone alive today in the United States who will eventually develop Alzheimer's disease is $47.1 trillion. The six drugs currently approved by the FDA for the treatment of Alzheimer's disease temporarily improve symptoms for some people, but have modest activity to slow or stop the progression of the disease and have unacceptable side effects for many. Approximately 491,000 new cases of AD and dementia are expected in 2020 for people aged 65 or older.

Diseases causing mild or greater cognitive impairment affect millions of patients across the globe. Alzheimer's disease and dementia in particular are devastating to those they affect. While there are treatments available on the market to aid with some of the early symptoms of Alzheimer's disease and dementia, there are no disease modifying treatments approved that have demonstrated a clinically significant slowing of MCI and AD progression, which presents a major unmet medical need.

Familial Alzheimer's Disease (FAD) is thought to be related to hereditary mutations that increase amyloid beta in the brain, which can cause plaques and neuroinflammation. This disease leads to symptoms in ages of 30's to 50's. Late Onset Alzheimer's Disease (LOAD) occurs in the elderly (typically over 55). While often described as Alzheimer's Disease and Related Dementias (ADRD), studies on the correlation between amyloid beta plaques in the brains of deceased subjects and cognitive decline show that there are plaques in brains of subjects that die without any sign of cognitive decline, and there are LOAD subjects that have no evidence of postmortem plaques indicating that amyloid beta pathology alone is not alone responsible for the disease. Nevertheless, the amyloid hypothesis has been widely adopted and used to guide pharmaceutical development and FDA guidance for AD drug and diagnostic test development. A consequence of the amyloid hypothesis is an assumption that amyloid (and/or p-tau) positivity distinguishes pathologically diverse dementia subject populations, each of which requiring an intervention with a mechanism of action directed against amyloid beta, p-tau, another molecular target for subjects lacking amyloid beta/p-tau positivity.

A large body of scientific evidence indicates that AD disease pathophysiology is driven by combinations of genetics, aging and aging-related metabolic inflammation. AD has a complex and incompletely understood etiology, but neuroinflammation is well accepted as an essential feature of the disease. The association of neuroinflammation with systemic inflammatory conditions is well documented. Such systemic inflammatory conditions include rheumatoid arthritis, inflammatory bowel disease and type 2 diabetes (T2D). AD association with T2D has been especially well documented and is frequently observed in the context of a constellation of symptoms indicative of lipid and glucose metabolism dysregulation in an inflamed host commonly known as metabolic syndrome. Systemic inflammatory mediators promote neuroinflammation through blood-brain barrier permeation and disruption of the blood-brain barrier to facilitate entry of inflammatory cells and otherwise impermeable factors. In the CNS resident microglia and astroglia respond to inflammatory stimuli to perpetuate an inflammatory environment, which is characterized by oxidative stress and insulin resistance, both of which are mutually inductive with inflammation in the CNS. AD pathophysiology has been closely linked to insulin resistance and oxidative stress, with inflammation being stoked by factors such as hyper-phosphorylated tau fibrils and amyloid beta oligomers, the formation of which in turn is driven by inflammation, thus creating a forward feeding cycle.

### SUMMARY OF THE DISCLOSURE

Disclosed herein are methods to treat, reduce, or ameliorate a neurodegenerative condition or disease in a subject, the methods comprising administering to subjects 17α-ethynylandrost-5-ene-3β,7β,17β-triol and at least one pharmaceutically acceptable excipient, wherein the subjects have a waist to hip ratio greater than approximately 0.80. In some embodiments, the neurodegenerative condition or disease is Alzheimer's disease. In some embodiments, the neurodegenerative condition or disease is dementia. In some embodiments, the subject experiences an improvement in memory recall. In some embodiments, the subject experiences an improvement in memory loss. In some embodiments, the subject experiences an improvement in cloudy memory. In some embodiments, the subject experiences an improvement in mild cognitive dysfunction. In some embodiments, the subjects have a clinical dementia rating ranging from approximately 0.5 to 1-2 or an MMSE score of 12-29. In some embodiments, the subjects have a mini mental state exam score ranging from approximately 14-29. In some embodiments, the subjects have a 5% to 75% loss of cognitive function in one or more cognitive domains compared to their function in mid-life or prior to disease onset. In some embodiments, subjects experience an improvement in overall cognitive function that restores 5% to 100% of the lost functional capability. In all embodiments, 17α-ethynylandrost-5-ene-3β,7β,17β-triol is administered to a subject to achieve a concentration in plasma greater than 0.1 ng/mL for the majority of time in a day for the majority of days being treated. In some embodiments, the 17α-ethynylandrost-5-ene-3β,7β,17β-triol is administered orally. In some embodiments, the 17α-ethynylandrost-5-ene-3β,7β,17β-triol is administered intravenously. In some embodiments 17α-ethynylandrost-5-ene-3β,7β,17β-triol is administered transmucosally. In some embodiments 17α-ethynylandrost-5-ene-3β,7β,17β-triol is administered transcutaneously. In some embodiments 17α-ethynylandrost-5-ene-3β,7β,17β-triol is administered by inhalation. In some embodiments, subjects have a waist to hip ratio greater than approximately 0.80 in females, 0.95 in Caucasian males, and 0.90 in Asian males. In some embodiments, the 17α-ethynylandrost-5-ene-3β,7β,17β-triol is a solid-state form of 17α-ethynylandrost-5-ene-3β,7β,17β-triol. In some embodiments, the solid-state form of 17α-ethynylandrost-5-ene-3β,7β,17β-triol is crystalline solvate of 17α-ethynylandrost-5-ene-3β,7β,17β-triol. In some embodiments, the crystalline solvate is crystalline methanolate 17α-ethynylandrost-5-ene-3β,7β,17β-trio. In some embodiments, the crystalline solvate is crystalline ethanolate 17α-ethynylandrost-5-ene-3β,7β,17β-triol. In some embodiments, the crystalline solvate is crystalline hydrate 17α-ethynylandrost-5-ene-3β,7β,17β-triol. In some embodiments, the crystalline solvate is Form III 17α-ethynylandrost-5-ene-3β,7β,17β-triol. In some embodiments, the crystalline solvate is Form IV 17α-ethynylandrost-5-ene-3β,7β,17β-triol. In some embodiments, the crystalline solvate is Form V 17α-ethynylandrost-5-ene-3β,7β,17β-triol. In some embodiments, the solid-state form of 17α-ethynylandrost-5-ene-3β,7β,17β-triol is amorphous 17α-ethynylandrost-5-ene-3β,7β,17β-triol. In some embodiments, the pharmaceutical composition contains less than about 3% by weight of impurities.

Some embodiments relate to a method to increase intracellular concentration of glutathione in the cerebral cortex, limbic system, cerebellum, or brain stem in a subject in need thereof. In some embodiments, the method includes identifying a subject having a waist to hip ratio greater than approximately 0.80 and administering to the subject 17α-ethynylandrost-5-ene-3β,7β,17β-triol and at least one pharmaceutically acceptable excipient.

Some embodiments relate to a method to increase blood flow in support of cognitive activity in the cerebral cortex, limbic system, cerebellum, or brain stem in a subject in need thereof. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient.

Some embodiments related to a method to treat, prevent or decrease TNF production or TNF activation in a subject in need thereof. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient.

Not all objectives mentioned in this specification are necessarily achieved in all embodiments disclosed and/or claimed herein.

### BRIEF DESCRIPTION OF THE DRAWINGS

FIGS. 1A-1L illustrate scores for patients administered 17α-ethynylandrost-5-ene-3β,7β,17β-triol.

FIGS. 2A-2J illustrate results of the neuroimaging study for patients administered 17α-ethynylandrost-5-ene-3β,7β,17β-triol.

FIGS. 3A-3L illustrate results of CSF biomarker analysis for patients administered 17α-ethynylandrost-5-ene-3β,7β,17β-triol.

FIGS. 4A-4J illustrate results of peripheral blood biomarker analysis for patients administered 17α-ethynylandrost-5-ene-3β,7β,17β-triol.

FIG. 5 illustrates results of the correlation of change from baseline in PHQ-9 and baseline ADAS-Cog12, Total QDRS and AD Composite Score.

FIG. 6 illustrates results of the correlation of change from baseline in PHQ-9 and baseline ADAS-Cog12, Total QDRS and AD Composite Score.

FIG. 7 illustrates results of the correlation of change from baseline in PHQ-9 with change in other parameters.

FIG. 8 illustrates results of total PQD-9 score, all enrolled subjects (top) and change from baseline 0-4 Subjects (bottom).

FIG. 9 illustrates results of total PQD-9 score, all enrolled subjects (top) and change from baseline 5-9 Subjects (bottom).

FIG. 10 illustrates results of total PQD-9 score, all enrolled subjects (top) and change from baseline 10-14 Subjects (bottom).

FIG. 11 illustrates results of total PQD-9 score, all enrolled subjects (top) and change from baseline 15-19 Subjects (bottom).

FIG. 12 illustrates results of total PQD-9 score, all enrolled subjects (top) and change from baseline 20-27 Subjects (bottom).

FIG. 13 illustrates a radar plot of PHD-9 of the mean change from baseline for each domain scores with subjects >=5 total PQD-9 score at baseline.

FIG. 14 illustrates a total PQD-9 score regression of all enrolled population.

### DETAILED DESCRIPTION

The following description provides context and examples, but should not be interpreted to limit the scope of the disclosure covered by the claims that follow in this specification or in any other application that claims priority to this specification. No single component or collection of components is essential or indispensable. For example, in some embodiments one or more variables, such as Y or Y and Q may be omitted. Any feature, structure, component, material, step, or method that is described and/or illustrated in any embodiment in this specification can be used with or instead of any feature, structure, component, material, step, or method that is described and/or illustrated in any other embodiment in this specification.

### Definitions

As used herein and unless otherwise stated or implied by context, terms that are used herein have the meanings that are defined here. The descriptions of embodiments and examples that are described illustrate the disclosure and they are not intended to limit it in any way. Unless otherwise contraindicated or implied, e.g., by including mutually exclusive elements or options, in these definitions and throughout this specification, the terms “a” and “an” mean one or more and the term “or” means and/or.

A “formulation” or the like means a composition that one can administer to a subject, e.g., human or animal. Formulations are suitable for human or veterinary applications and would typically have expected characteristics for the formulation, e.g., parenteral formulations for human use would usually be sterile solutions or suspensions.

An “excipient”, “carrier”, “pharmaceutically acceptable carrier” or similar terms mean one or more component(s) or ingredient(s) that is acceptable in the sense of being compatible with the other ingredients in the disclosed compositions or formulations and not overly deleterious to the patient, animal, tissues or cells to which the formulation is to be administered.

As used herein, “subject,” “host,” “patient,” and “individual” are used interchangeably and shall be given their ordinary meaning in the art and shall also refer to an organism that has cancer and/or leukemia. This includes mammals, e.g., a human, a non-human primate, ungulates, canines, felines, equines, mice, rats, and the like. The term “mammal” includes both human and non-human mammals.

The terms “therapeutically effective amount” and “effective amount” refer to the amount of active pharmaceutical ingredient necessary to provide the desired pharmacologic result. In practice, the therapeutically effective amount will vary widely depending on the severity of the disease condition, age of the subject, and the desired therapeutic effect.

The terms “treatment,” “treating,” “treat,” and the like shall be given their ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. The terms “treatment,” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Methods of Treatment

Aspects of the present disclosure relate to methods to prevent, treat, reduce, or ameliorate a neurodegenerative condition or disease. In some embodiments, a method for preventing, treating, reducing, or ameliorating a neurodegenerative condition or disease may include administering to a patient in need thereof an effective amount of a pharmaceutical composition. In some embodiments, the pharmaceutical composition includes a compound having the structure:

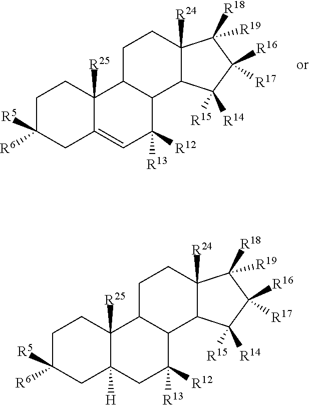

wherein, one of R⁵ and R⁶ is —OH and the other R⁵ and R⁶ is —H, one of R¹² and R¹³ is —OH and the other R¹² and R¹³ is —H, R¹⁴ and R¹⁵ are H, R¹⁶ is —H, R¹⁷ is —H or —OH, R¹⁸ is —OH, R¹⁹ is ethynyl, and R²⁴ and R²⁵ are —CH₃.

In some embodiments, the pharmaceutical composition includes a compound having the structure:

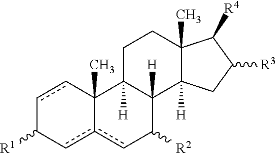

wherein, R¹ is —OH or an ester, R² is —OH or an ether, R³ is —OH, ═O, a halogen, an ester or ═CH₂, and R⁴ is an optionally substituted amine, an amide, an N-linked amino acid, ═NOH or —NHOH.

In some embodiments, the pharmaceutical composition includes an effective amount of a compound having the structure:

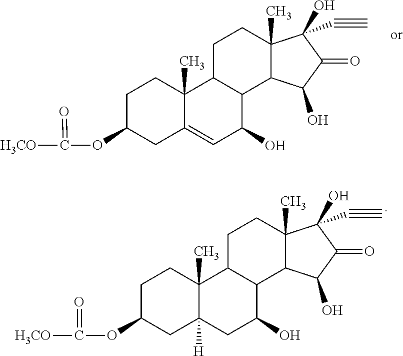 [SMILES: C#C[C@]1(O)C(=O)[C@@H](O)C2C3C(CC[C@@]21C)[C@@]1(C)CC[C@H](OC(=O)OC)CC1=C[C@@H]3O ; C#C[C@]1(O)C(=O)[C@@H](O)C2C3C(CC[C@@]21C)[C@@]1(C)CC[C@H](OC(=O)OC)C[C@@H]1C[C@@H]3O]

In some embodiments, the pharmaceutical composition includes an effective amount of a compound having the structure:

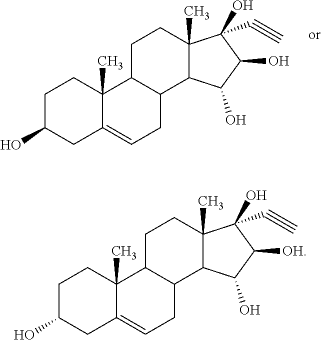 [SMILES: C#C[C@]1(O)[C@@H](O)[C@H](O)C2C3CC=C4C[C@@H](O)CC[C@]4(C)C3CC[C@@]21C ; C#C[C@]1(O)[C@@H](O)[C@H](O)C2C3CC=C4C[C@H](O)CC[C@]4(C)C3CC[C@@]21C]

In some embodiments, the pharmaceutical composition includes an effective amount of a compound having the structure:

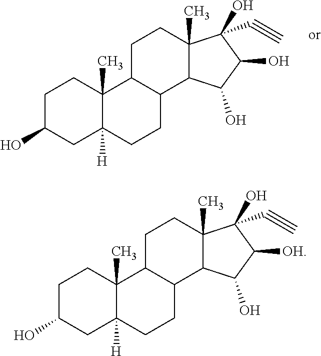 [SMILES: C#C[C@]1(O)[C@@H](O)[C@H](O)C2C3CC[C@H]4C[C@@H](O)CC[C@]4(C)C3CC[C@@]21C ; C#C[C@]1(O)[C@@H](O)[C@H](O)C2C3CC[C@H]4C[C@H](O)CC[C@]4(C)C3CC[C@@]21C]

In some embodiments, the pharmaceutical composition includes an effective amount of a compound having the structure:

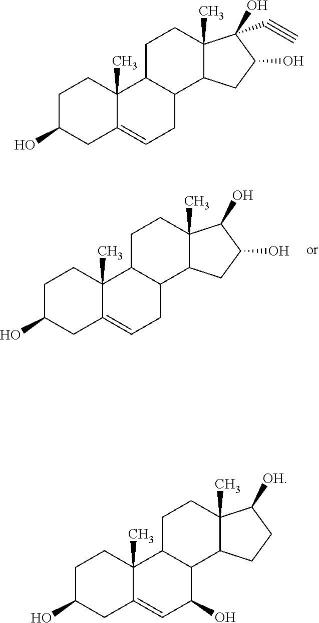 [SMILES: C#C[C@]1(O)[C@H](O)CC2C3CC=C4C[C@@H](O)CC[C@]4(C)C3CC[C@@]21C ; C[C@]12CC[C@H](O)CC1=CCC1C2CC[C@@]2(C)C1C[C@@H](O)[C@@H]2O ; C[C@]12CC[C@H](O)CC1=C[C@H](O)C1C2CC[C@@]2(C)C1CC[C@@H]2O]

In some embodiments, the pharmaceutical composition includes an effective amount of a compound having the structure:

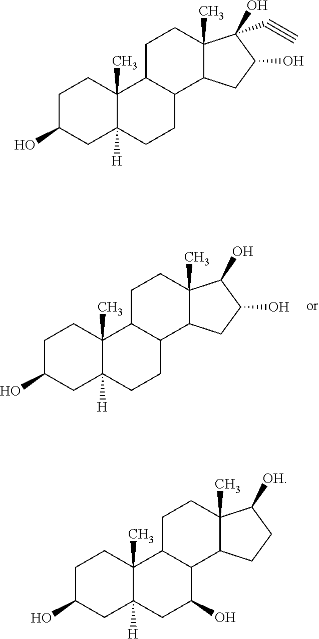 [SMILES: C#C[C@]1(O)[C@H](O)CC2C3CC[C@H]4C[C@@H](O)CC[C@]4(C)C3CC[C@@]21C ; C[C@]12CCC3C(CC[C@H]4C[C@@H](O)CC[C@]34C)C1C[C@@H](O)[C@@H]2O ; C[C@]12CCC3C(C1CC[C@@H]2O)[C@@H](O)C[C@H]1C[C@@H](O)CC[C@]31C]

In some embodiments, the pharmaceutical composition includes an effective amount of a compound having the structure:

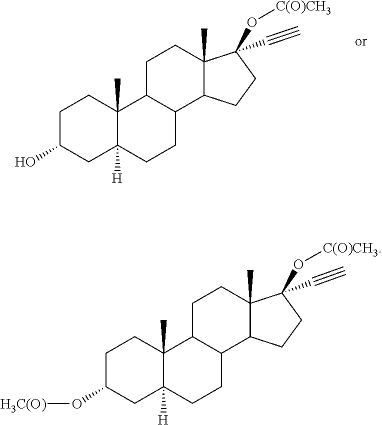

In some embodiments, the pharmaceutical composition includes an effective amount of a compound having the structure:

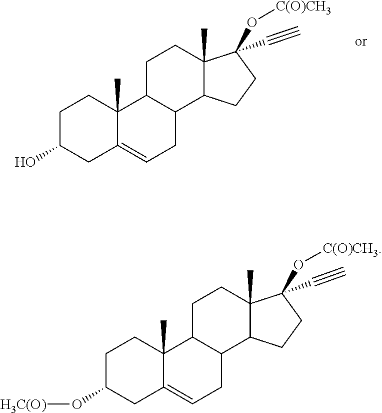

In some embodiments, the pharmaceutical composition includes 17-ethynyl-10R, 13S-dimethyl 2, 3, 4, 7, 8R, 9S, 10, 11, 12, 13, 14S, 15, 16, 17-hexadecahydro-1H-cyclopenta[a]phenanthrene-3R, 7R, 17S-triol, which is represented by Formula 1. The compound of Formula 1 is may also be referred to as Compound 1 or 17α-ethynylandrost-5-ene-3β,7β,17β-triol and is represented by the structure below.

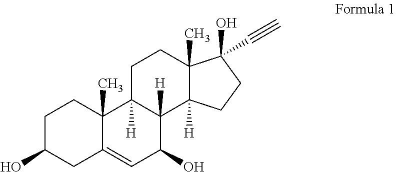

In some embodiments, the pharmaceutical composition includes (3S,5R,7S,8R,9S,10S,13S,14S,17R)-17-ethynyl-10,13-dimethylhexadecahydro-1H-cyclopenta[a]phenanthrene-3,7,17-triol, which is represented by Formula 2. The compound of Formula 2 may also be referred to as Compound 2, and is represented by the structure below.

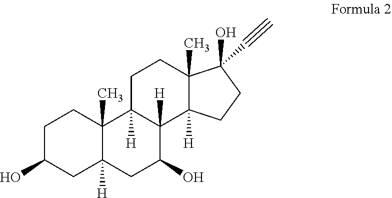

In some embodiments, the neurodegenerative condition or disease includes, but is not limited to, progressive central nervous system disorders, mild memory loss, early or late onset Alzheimer's disease, probable Alzheimer's disease, Alzheimer's disease related dementias, frontotemporal lobar dementia, Parkinson's disease, traumatic brain injury, post-operative cognitive dysfunction, clouded memory, mild memory recall issues, mild cognitive impairment, mild cognitive dysfunction, or mild cognitive decline. In some embodiments, the neurodegenerative condition or disease is mild cognitive impairment. In some embodiments, the mild cognitive impairment is Alzheimer's disease. In other embodiments, the mild cognitive impairment is dementia. In some embodiments, the neurodegenerative condition or disease is associated with memory. In some embodiments, the neurodegenerative condition or disease is difficulty with cognitive memory. In some embodiments, the neurodegenerative condition or disease is associated with clarity. In some embodiments, the neurodegenerative condition or disease is difficulty with cognitive clarity.

The clinical dementia rating is a numeric scale, ranging from 0 to 3, used to quantify the severity of symptoms of dementia. A zero indicates no symptoms, while a three indicates severe symptoms. The rating scale takes into account several factors, such as memory, orientation, community affairs, personal care, judgment, problem solving, and ability to partake in one's hobbies. In some embodiments, the clinical dementia rating for subjects is equal to or greater than approximately 0.5, 1, 2, 3, or ranges including and/or spanning the aforementioned values. For example, the clinical dementia rating for subjects may be greater than or equal to approximately 1, or may be greater than or equal to approximately 1 to 2.

In some embodiments, the subject may experience an improvement in overall cognitive function or an improvement in one or more specific domains of cognitive function, such as memory, clarity, learning, language, executive functions, complex attention, social cognition, and perceptual and motor functions. Overall cognitive function and domain specific function can be measured through exams such as the clinical dementia rating test or the mini mental state examination, Alzheimer's disease assessment scales of various lengths, Montreal Cognitive Assessment and similar instruments, tests that measure clinicians impression of change, such as Alzheimer's Disease Cooperative Study Clinical Global Impression of Change [ADCS CGIC], or tests that measure one's ability to function, such as Alzheimer's Disease Cooperative Study Activities of Daily Living (ADCS ADL). Improvements can also be observed through memory, orientation, community affairs, personal care, attention, calculation, registration, judgment, problem solving, recall, and language skills, or estimated from interviews with family members and caregivers. The tests that can be used to measure improved cognitive function are not limited to the afore mentioned examples. In some embodiments, overall cognitive function or function in a specific domain of function may be restored to an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of levels found in mid-life or prior to disease onset or ranges including and/or spanning the aforementioned values. For example, subjects may experience an improvement in overall or domain specific cognitive function that restores approximately 5% to 100% of their mid-life function or function before disease onset after administration of a compound as described herein (e.g., 17α-ethynylandrost-5-ene-3β,7β,17β-triol) and at least one pharmaceutically acceptable excipient. In some embodiments, subjects may an improvement in overall or domain specific cognitive function that restores approximately 5% to 100% after administration of a compound as described herein (e.g., 17α-ethynylandrost-5-ene-3β,7β,17β-triol) and at least one pharmaceutically acceptable excipient.

In some embodiments, the subject may experience an improvement in mild memory loss. In some embodiments, mild memory loss may include short-term memory. Short-term memory is the capacity for holding a small amount of information in an active, readily available state for a short interval. In some embodiments, the mild memory loss may be improved in a subject to approximately 5%, 10%, 15%, 20%, 25%, 30%, 35%, 40%, 45%, 50%, 55%, 60%, 65%, 70%, 75%, 80%, 85%, 90%, 99%, of levels found in mid-life or prior to disease onset or ranges including and/or spanning the aforementioned values. For example, subjects may experience an improvement from mild memory loss that restores approximately 5% to 100% of their mild memory loss before disease onset after administration of a compound as described herein (e.g., 17α-ethynylandrost-5-ene-3β,7β,17β-triol) and at least one pharmaceutically acceptable excipient. In some embodiments, subjects may an improvement in mild memory loss from approximately 5% to 100% after administration of a compound as described herein (e.g., 17α-ethynylandrost-5-ene-3β,7β,17β-triol) and at least one pharmaceutically acceptable excipient.

In some embodiments, the subject may experience an improvement in mild memory recall issues. In some embodiments, the mild memory recall issues loss may be improved in a subject by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of levels found in mid-life or prior to disease onset or ranges including and/or spanning the aforementioned values. For example, subjects may experience an improvement in mild memory recall issues that restores approximately 5% to 100% of their mild memory recall issues before disease onset after administration of a compound as described herein (e.g., 17α-ethynylandrost-5-ene-3β,7β,17β-triol) and at least one pharmaceutically acceptable excipient. In some embodiments, subjects may improve in mild memory recall from approximately 5% to 100% after administration of a compound as described herein (e.g., 17α-ethynylandrost-5-ene-3β,7β,17β-triol) and at least one pharmaceutically acceptable excipient.

In some embodiments, the subject may experience an improvement in cloudy memory. In some embodiments, cloudy memory may include brain fog or brain fog syndrome. In some embodiments, cloudy memory may include memory problems, a lack of mental clarity, poor concentration, and an inability to focus. In some embodiments, the cloudy memory may be improved in a subject by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of levels found in mid-life or prior to disease onset or ranges including and/or spanning the aforementioned values. For example, subjects may experience an improvement cloudy memory that restores approximately 5% to 100% of memory before disease onset after administration of a compound as described herein (e.g., 17α-ethynylandrost-5-ene-3β,7β,17β-triol) and at least one pharmaceutically acceptable excipient. In some embodiments, subjects may improve their cloudy memory from approximately 5% to 100% after administration of a compound as described herein (e.g., 17α-ethynylandrost-5-ene-3β,7β,17β-triol) and at least one pharmaceutically acceptable excipient.

In some embodiments, the subject may experience an improvement in mild cognitive dysfunction. In some embodiments, the mild cognitive dysfunction may be improved in a subject by an amount equal to or greater than approximately 5%, 10%, 15%, 20%, 25%, 30%, 35%, 40%, 45%, 50%, 55%, 60%, 65%, 70%, 75%, 80%, 85%, 90%, 99% of levels found in mid-life or prior to disease onset or ranges including and/or spanning the aforementioned values. For example, subjects may experience an improvement in mild cognitive dysfunction that restores 5% to 100% of their cognitive function before disease onset after administration of a compound as described herein (e.g., 17α-ethynylandrost-5-ene-3β,7β,17β-triol) and at least one pharmaceutically acceptable excipient. In some embodiments, subjects may improve their cognitive function from approximately 5% to 100% after administration of a compound as described herein (e.g., 17α-ethynylandrost-5-ene-3β,7β,17β-triol) and at least one pharmaceutically acceptable excipient.

In some embodiments, the subject may experience an improvement in mild cognitive decline. In some embodiments, the mild cognitive decline loss may be improved in a subject by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of levels found in mid-life or prior to disease onset or ranges including and/or spanning the aforementioned values. For example, subjects may experience an improvement from mild cognitive decline that restores approximately 5% to 100% of their cognitive function before disease onset after administration of a compound as described herein (e.g., 17α-ethynylandrost-5-ene-3β,7β,17β-triol) and at least one pharmaceutically acceptable excipient. In some embodiments, subjects may improve their mild cognitive decline from approximately 5% to 100% after administration of a compound as described herein (e.g., 17α-ethynylandrost-5-ene-3β,7β,17β-triol) and at least one pharmaceutically acceptable excipient.

In some embodiments, the subject may experience a level of cognitive impairment shown through a loss of cognitive function, either global or in specific domains. Cognitive function can be measured through exams as described herein such as the clinical dementia rating test or the mini mental state examination, or can be observed through memory, orientation, community affairs, personal care, attention, calculation, registration, judgment, problem solving, recall, and language skills. In some embodiments, cognitive impairment may be evident through a loss of cognitive function that is an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of function at mid-life or prior to disease onset or ranges including and/or spanning the aforementioned values. For example, subjects may experience an improvement from cognitive impairment that restores approximately 5% to 100% of their cognitive function before disease onset after administration of a compound as described herein (e.g., 17α-ethynylandrost-5-ene-3β,7β,17β-triol) and at least one pharmaceutically acceptable excipient. In some embodiments, subjects may improve their cloudy memory from approximately 5% to 100% after administration of a compound as described herein (e.g., 17α-ethynylandrost-5-ene-3β,7β,17β-triol) and at least one pharmaceutically acceptable excipient.

In some embodiments, the subject may experience a level of cognitive impairment shown through a loss of cognitive function and have a clinical dementia rating that indicates mild to severe symptoms of Alzheimer's Disease or dementia. In some embodiments, cognitive impairment may be evident through a loss of cognitive function that is an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of function at mid-life or prior to disease onset or ranges including and/or spanning the aforementioned values. For example, subjects may experience an improvement in cognitive function and have a clinical dementia rating that indicates mild to severe symptoms of Alzheimer's Disease or dementia from approximately 5% to 100% before disease onset after administration of a compound as described herein (e.g., 17α-ethynylandrost-5-ene-3β,7β,17β-triol) and at least one pharmaceutically acceptable excipient. In some embodiments, subjects may improve their cognitive function and have a clinical dementia rating that indicates mild to severe symptoms of Alzheimer's Disease or dementia from approximately 5% to 100% after administration of a compound as described herein (e.g., 17α-ethynylandrost-5-ene-3β,7β,17β-triol) and at least one pharmaceutically acceptable excipient.

In some embodiments, the subject may experience an improvement in overall cognitive function and have a clinical dementia rating that indicates mild to severe symptoms of dementia. In some embodiments, overall cognitive function may improve by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of function at mid-life or prior to disease onset or ranges including and/or spanning the aforementioned values. For example, subjects may experience an improvement in overall cognitive function and have a clinical dementia rating that indicates mild to severe symptoms of dementia from approximately 5% to 100% before disease onset. In some embodiments, subjects may improve overall cognitive function and have a clinical dementia rating that indicates mild to severe symptoms of dementia from approximately 5% to 100% after administration of a compound as described herein (e.g., 17α-ethynylandrost-5-ene-3β,7β,17β-triol) and at least one pharmaceutically acceptable excipient.

In some embodiments, the subject is at a higher risk for progressive cognitive impairment that is correlated with elevated waist-to-hip ratio (“WHR”). Elevated WHR may occur in the absence of increased body weight or increased BMI. The greater a waist to hip ratio is, the more visceral fat a subject carries. Greater amounts of visceral fat provide a systemic inflammatory influence that can lead to dysregulation of glucose and lipid metabolism, increased cholesterol, and higher blood pressure. Excess visceral fat can also be an indicator of a higher likelihood to have other health problems such as type 2 diabetes, heart disease, and stroke. In some embodiments, the waist to hip ratio for subjects is equal to or greater than approximately 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, or ranges including and/or spanning the aforementioned values. For example, the waist to hip ratio for a subject may be greater than or equal to approximately 0.80, or may be greater than or equal to approximately 0.95. In some embodiments, the subject is female. In some embodiments, the subject is male. In some embodiments, the subject is an Asian or Hispanic male or female. In some embodiments, the subject has a waist to hip ratio greater than approximately than 0.80 is female. In some embodiments, the subject has a waist to hip ratio greater than approximately 0.95 is male other than Asian or Hispanic. In some embodiments, the subject has a waist to hip ratio is greater than approximately 0.90 and the subject is Hispanic or Asian.

The mini mental state examination is a screening instrument for determining a subject's mental status through a set of 30 questions. The exam tests cognitive function by inquiring into registration, orientation, recall, language, calculation, and attention. The exam is scored on a 1-30 point scale. In some embodiments, the mini mental state exam score for subjects is equal to or greater than approximately 5, 6, 7, 8, 9, 10, 11, 12, 13, 14, 15, 16, 17, 18, 19, 20, 21, 22, 23, 24, 25, 26, 27, 28, 29, 30, or ranges including and/or spanning the aforementioned values. For example, subjects may have a mini mental state exam score ranging from approximately 12 to 29.

In some embodiments, the subject may experience a level of cognitive impairment shown through a loss of cognitive function and have a mini mental state examination score that indicates mild or moderate cognitive impairment. In some embodiments, cognitive impairment may be evident through a loss of cognitive function that is an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of their normal mid-life function or prior to disease onset or ranges including and/or spanning the aforementioned values, and the mini mental state exam score for subjects is equal to or greater than approximately 5, 6, 7, 8, 9, 10, 11, 12, 13, 14, 15, 16, 17, 18, 19, 20, 21, 22, 23, 24, 25, 26, 27, 28, 29, 30, or ranges including and/or spanning the aforementioned values. For example, subjects may have a mini mental state exam score ranging from approximately 12 to 29 and a level of cognitive impairment between approximately 5% and 75% loss of cognitive function and experience a level of cognitive impairment after administration of a compound as described herein (e.g., 17α-ethynylandrost-5-ene-3β,7β,17β-triol) and at least one pharmaceutically acceptable excipient.

In some embodiments, the subject may experience an improvement in overall cognitive function and have a mini mental state examination score that indicates mild or moderate cognitive impairment. In some embodiments, overall cognitive function may improve by an amount to restore approximately 5%, 10%, 15%, 20%, 25%, 30%, 35%, 40%, 45%, 50%, 55%, 60%, 65%, 70%, 75%, 80%, 85%, 90%, 99%, of their function or ranges including and/or spanning the aforementioned values, and the mini mental state exam score for subjects is equal to or greater than approximately 5, 6, 7, 8, 9, 10, 11, 12, 13, 14, 15, 16, 17, 18, 19, 20, 21, 22, 23, 24, 25, 26, 27, 28, 29, 30, or ranges including and/or spanning the aforementioned values. For example, subjects may have a mini mental state exam score ranging from approximately 12 to 29 and subjects may experience an improvement in overall cognitive function to restore approximately 5% to 100% of their normal mid-life or pre-disease onset function after administration of a compound as described herein (e.g., 17α-ethynylandrost-5-ene-3β,7β,17β-triol) and at least one pharmaceutically acceptable excipient.

Some embodiments relate to a method to increase intracellular concentration of glutathione in the cerebral cortex, limbic system, cerebellum, or brain stem in a subject in need thereof. In some embodiments, the method includes identifying a subject having a waist to hip ratio greater than approximately 0.80 and administering to the subject 17α-ethynylandrost-5-ene-3β,7β,17β-triol and at least one pharmaceutically acceptable excipient. In some embodiments, the intracellular concentration of glutathione in the cerebral cortex, limbic system, cerebellum, or brain stem in a subject in need thereof is increased by an amount equal to or greater than approximately 5%, 10%, 15%, 20%, 25%, 30%, 35%, 40%, 45%, 50%, 55%, 60%, 65%, 70%, 75%, 80%, 85%, 90%, 99% of levels found in mid-life or prior to disease onset or ranges including and/or spanning the aforementioned values. For example, subjects may experience an improvement in cerebral cortex, limbic system, cerebellum, or brain stem intracellular glutathione that restores 5% to 100% of their mid-life concentrations or concentration before disease onset after administration of a compound as described herein (e.g., 17α-ethynylandrost-5-ene-3β,7β,17β-triol) and at least one pharmaceutically acceptable excipient.

Some embodiments relate to a method to increase blood flow in support of cognitive activity in the cerebral cortex, limbic system, cerebellum, or brain stem in a subject in need thereof. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, the increase blood flow in support of cognitive activity in the cerebral cortex, limbic system, cerebellum, or brain stem in a subject in need thereof is increased by an amount equal to or greater than approximately 5%, 10%, 15%, 20%, 25%, 30%, 35%, 40%, 45%, 50%, 55%, 60%, 65%, 70%, 75%, 80%, 85%, 90%, 99% of levels found in mid-life or prior to disease onset or ranges including and/or spanning the aforementioned values. For example, subjects may experience an improvement in that restores 5% to 100% of their mid-life cerebral cortex, limbic system, cerebellum, or brain stem blood flow or cerebral cortex, limbic system, cerebellum, or brain stem blood flow before disease onset after administration of a compound as described herein (e.g., 17α-ethynylandrost-5-ene-3β,7β,17β-triol) and at least one pharmaceutically acceptable excipient. In some embodiments, a subject may experience an improvement of their mid-life cerebral cortex, limbic system, cerebellum, or brain stem blood flow or cerebral cortex, limbic system, cerebellum, or brain stem blood flow from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent or decrease TNF production or TNF activation in a subject in need thereof. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing or decreasing TNF production or TNF activation in the subject in need thereof is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99% of levels found in mid-life or prior to disease onset or ranges including and/or spanning the aforementioned values. For example, subjects may experience a decrease of TNF production or TNF activation that restores 5% to 100% of their mid-life concentrations or concentration before disease onset after administration of a compound as described herein (e.g., 17α-ethynylandrost-5-ene-3β,7β,17β-triol) and at least one pharmaceutically acceptable excipient. In some embodiments, a subject may experience a decrease of TNF production or TNF activation from approximately 5% to 100% after administration of a compound as described herein (e.g., 17α-ethynylandrost-5-ene-3β,7β,17β-triol) and at least one pharmaceutically acceptable excipient.

In some embodiments, administering to the subject a compound as described herein, for example, 17α-ethynylandrost-5-ene-3β,7β,17β-triol, decreases TNF production and TNF activation of a subjects TNFR1 receptor. In some embodiments, administering to the subject a compound as described herein, for example, 17α-ethynylandrost-5-ene-3β,7β,17β-triol, decreases, prevents, or ameliorates metabolic inflammation. In some embodiments, administering to the subject a compound as described herein treats, prevents, or ameliorates TNFR1 receptor phosphorylation in Ikk/MAP3K8/MEK dependent scaffolds. In some embodiments, administering to the subject 17α-ethynylandrost-5-ene-3β,7β,17β-triol treats, prevents, or ameliorates TNFR1 receptor phosphorylation in Ikk/MAP3K8/MEK dependent scaffolds.

Some embodiments relate to a method to treat, prevent, manage, ameliorate, or improve a psychological condition in a subject in need thereof. In some embodiments, the method includes identifying a subjecting with a psychological condition. In some embodiments, the method includes administering to the subject a therapeutically effective amount of a composition comprising a compound as described herein and at least one pharmaceutically acceptable excipient, thereby treating, managing, ameliorating, or improving the psychological condition. In some embodiments, the method includes administering to the subject a therapeutically effective amount of a composition comprising 17α-ethynylandrost-5-ene-3β,7β,17β-triol and at least one pharmaceutically acceptable excipient, thereby treating, managing, ameliorating, or improving the psychological condition. In some embodiments, the psychological condition is selected from the group consisting of depression, anxiety, low self-esteem, low motivation, apathy, mental clarity, attention disorders, disorders of executive function, cognitive engagement, obsessive compulsive disorder, feelings of hope, feelings of independence, or combinations thereof. In some embodiments, treating, managing, ameliorating, or improving the psychological condition of the subject improves one or more of mood, self-esteem, mental clarity, feelings of hope, and feelings of independence. In some embodiments, treating, managing, ameliorating, or improving the psychological condition of the subject decreases one or more of depression and apathy. In some embodiments, the subject experiences an improvement in memory loss. In some embodiments, subject experiences an improvement in cloudy memory. In some embodiments, the subject experiences an improvement in mild cognitive dysfunction. In some embodiments, the subject experiences an improvement in overall the psychological condition ranging from approximately 5% to 75%. In some embodiments, the subject experiences a decrease in overall the psychological condition ranging from approximately 5% to 75%. In some embodiments, the psychological condition may be treated, managed, ameliorated, or improved in a subject by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of levels found in mid-life or prior to disease onset or ranges including and/or spanning the aforementioned values. For example, subjects may experience an improvement in psychological condition that restores approximately 5% to 100% of their psychological condition or function before disease onset or prior to administration of the composition. In some embodiments, subjects may experience an improvement in psychological condition that restores approximately 5% to 100% of their psychological condition or function after administration of a compound as described herein (e.g., 17α-ethynylandrost-5-ene-3β,7β,17β-triol) and at least one pharmaceutically acceptable excipient.

Some embodiments relate to a method to treat, manage, ameliorate, or improve a behavioral condition in a subject in need thereof. In some embodiments, the method includes identifying a subjecting with a behavioral condition. In some embodiments, the method includes administering to the subject a therapeutically effective amount of a composition comprising 17α-ethynylandrost-5-ene-3β,7β,17β-triol and at least one pharmaceutically acceptable excipient, thereby treating, managing, ameliorating, or improving the behavioral condition. In some embodiments, the behavioral condition may be selected from the group consisting of, but not limited to, self-care, agitation, aggression, restlessness, wandering, physical outbursts, verbal outbursts, pacing, irritability, engagement, or combinations thereof. In some embodiments, treating, managing, ameliorating, or improving the behavioral condition of the subject improves one or more of self-care, agitation, aggression, restlessness, and engagement. In some embodiments, treating, managing, ameliorating, or improving the behavioral condition of the subject decreases one or more of agitation, aggression, restlessness, wandering, physical outbursts, verbal outbursts, pacing, and irritability. In some embodiments, subject experiences an improvement in overall the behavioral condition ranging from approximately 5% to 75%. In some embodiments, the subject experiences a decrease in overall the behavioral condition ranging from approximately 5% to 75%. In some embodiments, the behavioral condition may be treated, managed, ameliorated, or improved in a subject by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, of levels found in mid-life or prior to disease onset or ranges including and/or spanning the aforementioned values. For example, subjects may experience an improvement in behavioral condition that restores approximately 5% to 100% of their behavioral condition or function before disease onset or prior to administration of the composition. In some embodiments, subjects may experience an improvement in behavioral condition from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, manage, ameliorate, or improve a physical condition in a subject in need thereof. In some embodiments, the method includes identifying a subjecting with a physical condition. In some embodiments, the method includes administering to the subject a therapeutically effective amount of a composition comprising 17α-ethynylandrost-5-ene-3β,7β,17β-triol and at least one pharmaceutically acceptable excipient, thereby treating, managing, ameliorating, or improving the physical condition. In some embodiments, the physical condition is selected from the group consisting of weight loss, loss of motor coordination and balance, muscle weakness and stiffness, pain, chronic pain, headaches, sleep, movement, fatigue, sleep disturbance, eating disorders, or combinations thereof. In some embodiments, the treating, managing, ameliorating, or improving the physical condition of the subject improves one or more of weight, motor coordination and balance, muscle weakness and stiffness, energy, sleep, movement, and fatigue. In some embodiments, the treating, managing, ameliorating, or improving the physical condition decreases one or more of loss of weight, loss of balance, muscle weakness and stiffness, pain, chronic pain, headaches, sleep disturbances, and eating disorders. In some embodiments, the subject experiences an improvement in overall the physical condition ranging from approximately 5% to 75% after administration of a compound as described herein (e.g., 17α-ethynylandrost-5-ene-3β,7β,17β-triol) and at least one pharmaceutically acceptable excipient. In some embodiments, the subject experiences a decrease in overall the physical condition ranging from approximately 5% to 75% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate apathy in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating apathy in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from apathy from administration of a compound as described herein restores 5% to 100% of their pre-apathetic state before disease onset after administration of a compound as described herein (e.g., 17α-ethynylandrost-5-ene-3β,7β,17β-triol) and at least one pharmaceutically acceptable excipient. In some embodiments, a subject may experience a decrease in apathy from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate depression in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating depression in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their depression from administration of a compound as described herein and restore 5% to 100% of their pre-depressive state before disease onset after administration of a compound as described herein (e.g., 17α-ethynylandrost-5-ene-3β,7β,17β-triol) and at least one pharmaceutically acceptable excipient. In some embodiments, a subject may experience a decrease in depression from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate a sleep disturbance in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating a sleep disturbance in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their sleep disturbances from administration of a compound as described herein and restore 5% to 100% of their sleep state before disease onset after administration of a compound as described herein (e.g., 17α-ethynylandrost-5-ene-3β,7β,17β-triol) and at least one pharmaceutically acceptable excipient. In some embodiments, a subject may experience a decrease in sleep disturbances from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate fatigue in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating fatigue in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their fatigue from administration of a compound as described herein and restore 5% to 100% of their pre-fatigue state before disease onset after administration of a compound as described herein (e.g., 17α-ethynylandrost-5-ene-3β,7β,17β-triol) and at least one pharmaceutically acceptable excipient. In some embodiments, a subject may experience a decrease in fatigue from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate an eating disorder in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating an eating disorder in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their eating disorder from administration of a compound as described herein and restore 5% to 100% of their pre-eating disorder state before disease onset after administration of a compound as described herein (e.g., 17α-ethynylandrost-5-ene-3β,7β,17β-triol) and at least one pharmaceutically acceptable excipient. In some embodiments, a subject may experience a decrease in eating disorders from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate low self-esteem in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating low self-esteem in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their low self-esteem from administration of a compound as described herein and restore 5% to 100% of their pre-low self-esteem state before disease onset after administration of a compound as described herein (e.g., 17α-ethynylandrost-5-ene-3β,7β,17β-triol) and at least one pharmaceutically acceptable excipient. In some embodiments, a subject may experience a decrease in low self-esteem from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate poor concentration in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating poor concentration in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their low self-esteem from administration of a compound as described herein and restore 5% to 100% of their pre-poor concentration state before disease onset after administration of a compound as described herein (e.g., 17α-ethynylandrost-5-ene-3β,7β,17β-triol) and at least one pharmaceutically acceptable excipient. In some embodiments, a subject may experience a decrease in poor concentration from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate hypokinesia in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating hypokinesia in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their hypokinesia from administration of a compound as described herein and restore 5% to 100% of their pre-hypokinesia before disease onset after administration of a compound as described herein (e.g., 17α-ethynylandrost-5-ene-3β,7β,17β-triol) and at least one pharmaceutically acceptable excipient. In some embodiments, a subject may experience a decrease in hypokinesia from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate suicidal ideation in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating suicidal ideation in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their suicidal ideation from administration of a compound as described herein and restore 5% to 100% of their pre-suicidal ideation before disease onset after administration of a compound as described herein (e.g., 17α-ethynylandrost-5-ene-3β,7β,17β-triol) and at least one pharmaceutically acceptable excipient. In some embodiments, a subject may experience a decrease in suicidal ideation from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate a subject's mood in a subject in need thereof. In some embodiments, the subject's mood may be difficulty with their mood. In some embodiments, the subject's mood may be fear or worry of the future. In some embodiments, the subject's mood may impact their relationships. In some embodiments, the subject's mood may increase their dependence on others. In some embodiments, the subject's mood may cause them to have difficulty with their work. In some embodiments, the subject's mood may cause the subject to have difficulty with engagement. In some embodiments, the subject's mood may cause the subject to have difficulty with self-care. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating a subject's mood in a subject is increased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their mood from administration of a compound as described herein and restore 5% to 100% of their pre-mood before disease onset after administration of a compound as described herein (e.g., 17α-ethynylandrost-5-ene-3β,7β,17β-triol) and at least one pharmaceutically acceptable excipient. In some embodiments, a subject may experience an increase in their mood from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate numbness in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating numbness in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their numbness from administration of a compound as described herein and restore 5% to 100% of their pre-numbness before disease onset after administration of a compound as described herein (e.g., 17α-ethynylandrost-5-ene-3β,7β,17β-triol) and at least one pharmaceutically acceptable excipient. In some embodiments, a subject may experience a decrease in numbness from approximately 5% to 100% after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate neuropathy in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating neuropathy in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement from their neuropathy from administration of a compound as described herein and restore 5% to 100% of their pre-numbness before disease onset after administration of a compound as described herein (e.g., 17α-ethynylandrost-5-ene-3β,7β,17β-triol) and at least one pharmaceutically acceptable excipient. In some embodiments, a subject may experience a decrease in neuropathy from approximately 5% to 100% after administration of a compound as described herein (e.g., 17α-ethynylandrost-5-ene-3β,7β,17β-triol) and at least one pharmaceutically acceptable excipient.

Some embodiments relate to a method to reduce weight or cause weight loss in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, reducing or causing weight loss in a subject is a decrease in the mass of a subject from an amount equal to or greater than approximately 5%, 10%, 15%, 20%, 25%, 30%, 35%, 40%, 45%, 50%, or ranges including and/or spanning the aforementioned values. For example, subjects may experience improvement to their weight from administration of a compound as described herein and restore 5% to 50% of their pre-weight gain before disease onset after administration of a compound as described herein (e.g., 17α-ethynylandrost-5-ene-3β,7β,17β-triol) and at least one pharmaceutically acceptable excipient. In some embodiments, a subject may experience a reduction in weight or weight loss from approximately 5% to 50% of a subject's mass after administration of a compound as described herein (e.g., 17α-ethynylandrost-5-ene-3β,7β,17β-triol) and at least one pharmaceutically acceptable excipient.

Some embodiments relate to a method to treat, prevent, reduce, or ameliorate loss of movement in a subject in need thereof. Some embodiments relate to a method to improve movement in a subject in need thereof. In some embodiments, the method includes administering to the subject a compound as described herein and at least one pharmaceutically acceptable salt. In some embodiments, the subject has a neurodegenerative disease or condition. In some embodiments, the neurodegenerative disease or condition is mild or moderate dementia. In some embodiments, the subjects have a waist to hip ratio greater than approximately 0.80. In some embodiments, the method includes identifying a subject having a waist to hip ratio greater than approximately 0.80, and administering to the subject 17α-ethynylandrost-5-ene-3β,7β,17β-triol and at least one pharmaceutically acceptable excipient. In some embodiments, treating, preventing, reducing, or ameliorating loss of movement or movement loss in a subject is decreased by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, or ranges including and/or spanning the aforementioned values. For example, subjects may experience improvement in their movement from administration of a compound as described herein and restore 5% to 100% of their pre-movement ability before disease onset after administration of a compound as described herein (e.g., 17α-ethynylandrost-5-ene-3β,7β,17β-triol) and at least one pharmaceutically acceptable excipient. In some embodiments, a subject may experience an improvement in movement from approximately 5% to 100% after administration of a compound as described herein (e.g., 17α-ethynylandrost-5-ene-3β,7β,17β-triol) and at least one pharmaceutically acceptable excipient.

In some embodiments, a pharmaceutical composition includes a compound as described herein and a pharmaceutically acceptable salt. In some embodiments, the pharmaceutical composition comprises a pharmaceutically acceptable form a compound as described herein. In some embodiments, the pharmaceutical composition includes a solid state form of a compound as described herein. In several embodiments, the pharmaceutical compositions include a solid state form of 17α-ethynylandrost-5-ene-3β,7β,17β-triol. In several embodiments, the solid state form is crystalline 17α-ethynylandrost-5-ene-3β,7β,17β-triol. In several embodiments, the solid state form is crystalline 17α-ethynylandrost-5-ene-3β,7β,17β-triol substantially free of 17α-ethynylandrost-5-ene-3β,7β,17β-triol in amorphous form. In several embodiments, the pharmaceutical composition contains less than about 3% by weight of impurities. In several embodiments, the pharmaceutical composition contains less than about 5% by weight of 3β-hydroxy-androst-5-ene-7,17-dione. In several embodiments, the pharmaceutical composition includes a pharmaceutically acceptable formulation of 17α-ethynylandrost-5-ene-3β,7β,17β-triol.

In several embodiments, the solid state form is crystalline solvate 17α-ethynylandrost-5-ene-3β,7β,17β-triol. In several embodiments, the crystalline solvate is crystalline methanolate 17α-ethynylandrost-5-ene-3β,7β,17β-triol. In several embodiments, the crystalline solvate is crystalline ethanolate 17α-ethynylandrost-5-ene-3β,7β,17β-triol. In several embodiments, the crystalline solvate is crystalline hydrate 17α-ethynylandrost-5-ene-3β,7β,17β-triol.

In several embodiments, the crystalline solvate is Form III 17α-ethynylandrost-5-ene-3β,7β,17β-triol. In several embodiments, the crystalline solvate is Form IV 17α-ethynylandrost-5-ene-3β,7β,17β-triol. In several embodiments, the crystalline solvate is Form V 17α-ethynylandrost-5-ene-3β,7β,17β-triol.

In several embodiments, the solid state form of 17α-ethynylandrost-5-ene-3β,7β,17β-triol is amorphous 17α-ethynylandrost-5-ene-3β,7β,17β-triol. In several embodiments, the amorphous 17α-ethynylandrost-5-ene-3β,7β,17β-triol substantially free of 17α-ethynylandrost-5-ene-3β,7β,17β-triol in solid state form.

In some embodiments, 17α-ethynylandrost-5-ene-3β,7β,17β-triol is administered orally. In other embodiments, 17α-ethynylandrost-5-ene-3β,7β,17β-triol is administered intravenously. In other embodiments, 17α-ethynylandrost-5-ene-3β,7β,17β-triol is administered topically.

In some embodiments, 17α-ethynylandrost-5-ene-3β,7β,17β-triol is administered as a formulation with at least one pharmaceutically acceptable excipient. In some embodiments, 17α-ethynylandrost-5-ene-3β,7β,17β-triol is administered as a formulation with at least one pharmaceutically acceptable excipient and at least one pharmaceutically acceptable carrier. In some embodiments, 17α-ethynylandrost-5-ene-3β,7β,17β-triol is administered as a formulation with at least one pharmaceutically acceptable carrier. Other pharmaceutically acceptable excipients suitable for use in the compositions include absorption enhancing agents, acidifying agents, agents for modified release, alkalizing agents, antioxidants, buffering agents, chelating agents, coloring agents, complexing agents, emulsifying agents, flavoring agents, humectants, humidity-adjusting agents, pH-adjusting agents, preservatives, solubilizing agents, stabilizers, surface-active agents, suspending agents, sweetening agents, taste-masking agents, and wetting agents.

Formulations include compositions comprising 1, 2, 3, 4 or more pharmaceutically acceptable excipients or carriers. The compositions are used to prepare formulations suitable for human or animal use. Suitable administration routes for formulations include oral, rectal, nasal, transmucosal, topical (including buccal and sublingual), vaginal, rectal and parenteral (including subcutaneous, intramuscular, intravenous, intradermal, intrathecal, intraocular and epidural). In general, aqueous and non-aqueous liquid or cream formulations are delivered by a parenteral, oral or topical route. In other embodiments, such as the disclosure intermittent dosing methods, 17α-ethynylandrost-5-ene-3β,7β,17β-triol may be present as an aqueous or a non-aqueous liquid formulation or a solid formulation suitable for administration by any of the routes disclosed herein, e.g., oral, topical, buccal, sublingual, parenteral, inhaled aerosol or a depot such as a subcutaneous depot or an intraperitoneal or intramuscular depot. It will be appreciated that the preferred route may vary with, for example, the subject's pathological condition or weight or the subject's response to therapy with 17α-ethynylandrost-5-ene-3β,7β,17β-triol or other therapy that is used or that is appropriate to the circumstances.

The formulations include those suitable for the foregoing administration routes. The formulations may conveniently be presented in unit dosage form and may be prepared by any of the methods known in the art of pharmacy. Techniques, excipients and formulations generally are found in, e.g., Remington's Pharmaceutical Sciences, Mack Publishing Co., Easton, Pa. 2022, 23^{rd} edition, Adeboye et al., PDA J. Pharm. Sci. Tech. 1997 51:166-171, G. Cole, et al., editors, Pharmaceutical Coating Technology, 1995, Taylor & Francis, ISBN 0 136628915, H. A. Lieberman, et al., editors, Pharmaceutical Dosage Forms, 1992 2^{nd} revised edition, volumes 1 and 2, Marcel Dekker, ISBN 0824793870, J. T. Carstensen. Pharmaceutical Preformulation, 1998, pages 1-306, Technomic Publishing Co. ISBN 1566766907. Exemplary excipients for formulations include emulsifying wax, propyl gallate, citric acid, lactic acid, polysorbate 80, sodium chloride, isopropyl palmitate, glycerin, white petrolatum and other excipients disclosed herein.

Formulations, or compositions disclosed herein for use to make formulations suitable for administration by the routes disclosed herein optionally comprise an average particle size in the range of about 0.01 to about 500 microns, about 0.1 to about 100 microns or about 0.5 to about 75 microns. Average particle sizes include a range between 0.01 and 500 microns in 0.05 micron or in 0.1 micron or other increments, e.g., an average particle size of about 0.05, 0.1, 0.5, 1, 1.5, 2.0, 2.5, 3.0, 3.5, 4.0, 4.5, 5.0, 5.5, 6, 7, 8, 9, 10, 15, 20, 25, 30, 35, 40, 50, 60, 75, 85, 100, 120, etc. microns). When 17α-ethynylandrost-5-ene-3β,7β,17β-triol or compositions that comprise 17α-ethynylandrost-5-ene-3β,7β,17β-triol are used as intermediates to make a formulation, they may comprise one, two, three or more of these average particle sizes, or size ranges. In preparing any of the compositions or formulations that are disclosed herein and that comprise 17α-ethynylandrost-5-ene-3β,7β,17β-triol (and optionally one or more excipients and/or one or more carriers), one may optionally mill, sieve or otherwise granulate the compound or composition to obtain a desired particle size.

Non-limiting examples of fillers suitable for use in the compositions include lactose, microcrystalline cellulose, hydroxypropylcellulose, hydroxypropyl methylcellulose, methyl cellulose polymers hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, and collagen.

Non-limiting examples of diluents suitable for use in the compositions include, for example, calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, and sugar.

Non-limiting examples of disintegrants suitable for use in the compositions include alginic acid or alginates, microcrystalline cellulose, low-substituted hydroxypropyl cellulose and other cellulose derivatives, croscarmellose sodium, crospovidone, polacrillin potassium, sodium starch glycolate, starch, pregelatinized starch, and carboxymethyl starch.

Non-limiting examples of binders suitable for use in the compositions include acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, polyethylene oxides, povidone, and pregelatinized starch.

Non-limiting examples of glidants and/or lubricants suitable for use in the compositions include stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, and sodium acetate.

Non-limiting examples of antioxidants suitable for use in the compositions include ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, and derivatives of tocopherol.

In several embodiments, the pharmaceutically acceptable excipient is selected from sodium dodecyl sulfate, microcrystalline cellulose, magnesium stearate, and any combination of the foregoing. In several embodiments, the pharmaceutically acceptable excipient is sodium dodecyl sulfate.

In several embodiments, the pharmaceutical compositions are formulated into oral dosage forms. In several embodiments, the dosage forms can include capsules and tablets. In some embodiments, the dosage forms can include one or more different types of delayed release layers selected from sealant and/or enteric layers. For example, delayed release layers having different release rate characteristics can provide the dosage form with different overall drug release characteristics. In some such embodiments, the pharmaceutically acceptable excipient is a surface active agent. In several embodiments, the surface active agent is present in an amount sufficient to provide 90% dissolution of the pharmaceutical composition in water at ambient temperature after 30 min. In several embodiments, the surface active agent is sodium lauryl sulfate. In several embodiments, the pharmaceutical composition is a capsule or a tablet.

In several embodiments, the use is concurrent with a use of at least one additional medicament. In several embodiments, the additional medicament is administered at a delay time after a first administration of the composition. In several embodiments, the first administration may occur using a dosage schedule that is daily, weekly, monthly, or any combination of the foregoing. In several embodiments, the dosage schedule of the first administration may include one, two, three or more daily dosages of the composition. In several embodiments, the dosage schedule of the first administration may include one, two, three or more weekly dosages of the composition. In several embodiments, the dosage schedule of the first administration may include one, two, three or more monthly dosages of the composition. In several embodiments, the delay time is equal to or greater than about: 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the delay time is equal to or greater than 2 years. In some embodiments, the delay time is zero and the additional medicament is administered concurrently with the first administration of the composition. In several embodiments, the additional medicament is administered using a dosage schedule that is daily, weekly, monthly, or any combination of the foregoing. In several embodiments, the dosage schedule of the additional medicament may include one, two, three or more daily dosages of the composition. In several embodiments, the dosage schedule of the additional medicament may include one, two, three or more weekly dosages of the composition. In several embodiments, the dosage schedule of the additional medicament may include one, two, three or more monthly dosages of the composition.

Several embodiments of the present disclosure relate to the use of 17α-ethynylandrost-5-ene-3β,7β,17β-triol in the manufacture of a medicament for treating a neurodegenerative condition.

Accordingly, some aspects described relate to the following numbered alternatives:

1. A method to treat, reduce or ameliorate of a neurodegenerative condition or disease in a subject in need thereof, the method comprising: identifying a subject having a waist to hip ratio greater than approximately 0.80; and administering to the subject a therapeutically effective amount of a composition comprising 17α-ethynylandrost-5-ene-3β,7β,17β-triol and at least one pharmaceutically acceptable excipient.

2. The method of alternative 1, wherein the subject having a waist to hip ratio greater than approximately than 0.80 is female.

3. The method of alternative 1, wherein the subject having a waist to hip ratio greater than approximately 0.95 is male other than Asian or Hispanic.

4. The method of alternative 1, wherein the subject having a waist to hip ratio is greater than approximately 0.90 and the subject is Hispanic or Asian.

5. The method of any one of alternatives 1 to 4, wherein the neurodegenerative condition is a mild or moderate cognitive impairment.

6. The method of alternative 5, wherein the mild or moderate cognitive impairment is late onset Alzheimer's disease or Alzheimer's disease, or probable Alzheimer's disease.

7. The method of any one of alternatives 1 to 5, wherein the neurodegenerative condition or disease is dementia.

8. The method of alternative 7, wherein the subject has a clinical dementia rating ranging from approximately 0.5-2.

9. The method of any one of alternatives 1 to 8, wherein the subject has a level of cognitive impairment between approximately 5% and 75% loss of cognitive function.

10. The method any one of alternatives 1 to 9, wherein the subject experiences an improvement in overall cognitive function.

11. The method of alternative 10, wherein the improvement in overall cognitive function is from about 5% to about 75%.

12. The method any one of alternatives 1 to 11, wherein the subject experiences an improvement in memory recall.

13. The method of alternative 12, wherein the improvement in memory recall is from about 5% to about 75%.

14. The method any one of alternatives 1 to 13, wherein the subject experiences an improvement in memory loss.

15. The method of alternative 14, wherein the improvement in memory loss is from about 5% to about 75%.

16. The method any one of alternatives 1 to 15, wherein the subject experiences an improvement in cloudy memory.

17. The method of alternative 16, wherein the improvement in cloudy memory is from about 5% to about 75%.

18. The method any one of alternatives 1 to 17, wherein the subject experiences an improvement in mild cognitive dysfunction.

19. The method of alternative 18, wherein the improvement in mild or moderate cognitive dysfunction ranging is from about 5% to about 75%.

20. The method of any one of alternatives 1 to 19, wherein the subject has a mini mental state exam score ranging from approximately 12-29.

21. The method of any one of alternatives 1 to 20, wherein the 17α-ethynylandrost-5-ene-3β,7β,17β-triol is administered orally.

22. The method of any one of alternatives 1 to 20, wherein the 17α-ethynylandrost-5-ene-3β,7β,17β-triol is administered intravenously.

23. The method of any one of alternatives 1 to 22, wherein the subject has a waist to hip ratio greater than or equal to approximately 0.90.

24. The method of any one of alternatives 1 to 22, wherein the subject has a waist to hip ratio greater than or equal to approximately 0.95.

25. The method of any one of Alternatives 1 to 24, wherein the 17α-ethynylandrost-5-ene-3β,7β,17β-triol is a solid state form of 17α-ethynylandrost-5-ene-3β,7β,17β-triol.

26. The method of Alternative 25, wherein the solid state form of 17α-ethynylandrost-5-ene-3β,7β,17β-triol is crystalline solvate of 17α-ethynylandrost-5-ene-3β,7β,17β-triol.

27. The method of Alternative 26, wherein the crystalline solvate is crystalline methanolate 17α-ethynylandrost-5-ene-3β,7β,17β-triol.

28. The method of Alternative 27, wherein the crystalline solvate is crystalline ethanolate 17α-ethynylandrost-5-ene-3β,7β,17β-triol.

29. The method of Alternative 28, wherein the crystalline solvate is crystalline hydrate 17α-ethynylandrost-5-ene-3β,7β,17β-triol.

30. The method of Alternative 28, wherein the crystalline solvate is Form III 17α-ethynylandrost-5-ene-3β,7β,17β-triol.

31. The method of Alternative 28, wherein the crystalline solvate is Form IV 17α-ethynylandrost-5-ene-3β,7β,17β-triol.

32. The method of Alternative 28, wherein the crystalline solvate is Form V 17α-ethynylandrost-5-ene-3β,7β,17β-triol.

33. The method of Alternative 27, wherein the solid state form of 17α-ethynylandrost-5-ene-3β,7β,17β-triol is amorphous 17α-ethynylandrost-5-ene-3β,7β,17β-triol.

34. The method of any one of Alternatives 1 to 33, wherein the composition contains less than about 3% by weight of impurities.

35. A method to increase intracellular concentration of glutathione in the cerebral cortex or limbic system in a subject in need thereof, the method comprising: identifying a subject having a waist to hip ratio greater than approximately 0.80; and administering to the subject a therapeutically effective amount of a composition comprising 17α-ethynylandrost-5-ene-3β,7β,17β-triol and at least one pharmaceutically acceptable excipient.

36. The method of alternative 35, wherein the subject having a waist to hip ratio greater than approximately than 0.80 is female.

37. The method of alternative 35, wherein the subject having a waist to hip ratio greater than approximately 0.95 is male other than Asian or Hispanic.

38. The method of alternative 35, wherein the subject having a waist to hip ratio is greater than approximately 0.90 and the subject is Hispanic or Asian.

39. The method of any one of alternatives 35 to 38, wherein the subject increases an intracellular concentration of glutathione in the cerebral cortex, limbic system, cerebellum, or brain stem by approximately 5% to 100%.

40. The method of alternative 35 or 39, wherein the subject experiences an improvement in overall cognitive function.

41. The method of alternative 40, wherein the subject experiences an improvement in overall cognitive function ranging from approximately 5% to 100% of their mid-life cognitive function or function before disease onset.

42. The method of any one of alternatives 35 to 41, wherein the subject experiences an improvement in memory recall.

43. The method of alternative 42, wherein the subject experiences an improvement in memory recall ranging from approximately 5% to 100% of their mid-life cognitive function or function before disease onset.

44. The method any one of alternatives 35 to 43, wherein the subject experiences an improvement in memory loss.

45. The method of alternative 44, wherein the subject experiences an improvement in memory loss ranging from approximately 5% to 100% of their mid-life cognitive function or function before disease onset.

46. The method any one of alternatives 35 to 45, wherein the subject experiences an improvement in cloudy memory.

47. The method of alternative 46, wherein the subject experiences an improvement in cloudy memory ranging from approximately 5% to 100% of their mid-life cognitive function or function before disease onset.

48. The method any one of alternatives 35 to 47, wherein the subject experiences an improvement in mild cognitive dysfunction.

49. The method of alternative 48, wherein the subject experiences an improvement in mild cognitive dysfunction ranging from approximately 5% to 100% of their mid-life cognitive function or function before disease onset.

50. The method of any one of alternatives 35 to 49, wherein the subject having a mini mental state exam score ranging from approximately 12-29.

51. The method of any one of alternatives 35 to 50, wherein the 17α-ethynylandrost-5-ene-3β,7β,17β-triol is administered orally.

52. The method of any one of alternatives 35 to 51, wherein the 17α-ethynylandrost-5-ene-3β,7β,17β-triol is administered intravenously.

53. The method of any one of alternatives 35 to 52, wherein the subject has a waist to hip ratio greater than or equal to approximately 0.90.

54. The method of any one of alternatives 35 to 53, wherein the subject has a waist to hip ratio greater than or equal to approximately 0.95.

55. The method of any one of Alternatives 35 to 54, wherein the 17α-ethynylandrost-5-ene-3β,7β,17β-triol is a solid state form of 17α-ethynylandrost-5-ene-3β,7β,17β-triol.

56. The method of Alternative 55, wherein the solid state form of 17α-ethynylandrost-5-ene-3β,7β,17β-triol is crystalline solvate of 17α-ethynylandrost-5-ene-3β,7β,17β-triol.

57. The method of Alternative 56, wherein the crystalline solvate is crystalline methanolate 17α-ethynylandrost-5-ene-3β,7β,17β-triol.

58. The method of Alternative 57, wherein the crystalline solvate is crystalline ethanolate 17α-ethynylandrost-5-ene-3β,7β,17β-triol.

59. The method of Alternative 56, wherein the crystalline solvate is crystalline hydrate 17α-ethynylandrost-5-ene-3β,7β,17β-triol.

60. The method of Alternative 56, wherein the crystalline solvate is Form III 17α-ethynylandrost-5-ene-3β,7β,17β-triol.

61. The method of Alternative 56, wherein the crystalline solvate is Form IV 17α-ethynylandrost-5-ene-3β,7β,17β-triol.

62. The method of Alternative 56, wherein the crystalline solvate is Form V 17α-ethynylandrost-5-ene-3β,7β,17β-triol.

63. The method of Alternative 55, wherein the solid state form of 17α-ethynylandrost-5-ene-3β,7β,17β-triol is amorphous 17α-ethynylandrost-5-ene-3β,7β,17β-triol.

64. The method of any one of Alternatives 35 to 63, wherein the pharmaceutical composition contains less than about 3% by weight of impurities.

65. A method to increase blood flow in support of cognitive activity in the cerebral cortex, limbic system, cerebellum, or brain stem in a subject in need thereof, the method comprising: identifying a subject having a waist to hip ratio greater than approximately 0.80; and administering to the subject a therapeutically effective amount of a composition comprising 17α-ethynylandrost-5-ene-3β,7β,17β-triol and at least one pharmaceutically acceptable excipient.

66. The method of alternative 65, wherein the subject has a waist to hip ratio greater than approximately than 0.80 is female.

67. The method of alternative 65, wherein the subject has a waist to hip ratio greater than approximately 0.95 is male other than Asian or Hispanic.

68. The method of alternative 65, wherein the subject has a waist to hip ratio is greater than approximately 0.90 and the subject is Hispanic or Asian.

69. The method of any one of alternatives 65 to 68, wherein the subject increases intracellular concentration of glutathione in the cerebral cortex, limbic system, cerebellum, or brain stem by approximately 5% to 100% of their mid-life cognitive function or function before disease onset.

70. The method of alternative 65 to 69, wherein the subject experiences an improvement in overall cognitive function.

71. The method of alternative 65, wherein the subject experiences an improvement in overall cognitive function ranging from approximately 5% to 100% of their mid-life cognitive function or function before disease onset.

72. The method any one of alternatives 65 to 71, wherein the subject experiences an improvement in memory recall.

73. The method of alternative 72, wherein the subject experiences an improvement in memory recall ranging from approximately 5% to 100% of their mid-life cognitive function or function before disease onset.

74. The method any one of alternatives 65 to 73, wherein the subject experiences an improvement in memory loss.

75. The method of alternative 74, wherein the subject experiences an improvement in memory loss ranging from approximately 5% to 100% of their mid-life cognitive function or function before disease onset.

76. The method any one of alternatives 65 to 75, wherein the subject experiences an improvement in cloudy memory.

77. The method of alternative 76, wherein the subject experiences an improvement in cloudy memory ranging from approximately 5% to 100% of their mid-life cognitive function or function before disease onset.

78. The method any one of alternatives 65 to 77, wherein the subject experiences an improvement in mild cognitive dysfunction.

79. The method of alternative 78, wherein the subject experiences an improvement in mild cognitive dysfunction ranging from approximately 5% to 100% of their mid-life cognitive function or function before disease onset.

80. The method of any one of alternatives 65 to 79, wherein the subject have a mini mental state exam score ranging from approximately 12-29.

81. The method of any one of alternatives 65 to 80, wherein the 17α-ethynylandrost-5-ene-3β,7β,17β-triol is administered orally.

82. The method of any one of alternatives 65 to 81, wherein the 17α-ethynylandrost-5-ene-3β,7β,17β-triol is administered intravenously.

83. The method of any one of alternatives 65 to 82, wherein the subject having a waist to hip ratio greater than or equal to approximately 0.90.

84. The method of any one of alternatives 65 to 83, wherein the subject having a waist to hip ratio greater than or equal to approximately 0.95.

85. The method of any one of Alternatives 65 to 84, wherein the 17α-ethynylandrost-5-ene-3β,7β,17β-triol is a solid state form of 17α-ethynylandrost-5-ene-3β,7β,17β-triol.

86. The method of Alternative 85, wherein the solid state form of 17α-ethynylandrost-5-ene-3β,7β,17β-triol is crystalline solvate of 17α-ethynylandrost-5-ene-3β,7β,17β-triol.

87. The method of Alternative 86, wherein the crystalline solvate is crystalline methanolate 17α-ethynylandrost-5-ene-3β,7β,17β-triol.

88. The method of Alternative 87, wherein the crystalline solvate is crystalline ethanolate 17α-ethynylandrost-5-ene-3β,7β,17β-triol.

89. The method of Alternative 88, wherein the crystalline solvate is crystalline hydrate 17α-ethynylandrost-5-ene-3β,7β,17β-triol.

90. The method of Alternative 88, wherein the crystalline solvate is Form III 17α-ethynylandrost-5-ene-3β,7β,17β-triol.

91. The method of Alternative 88, wherein the crystalline solvate is Form IV 17α-ethynylandrost-5-ene-3β,7β,17β-triol.

92. The method of Alternative 88, wherein the crystalline solvate is Form V 17α-ethynylandrost-5-ene-3β,7β,17β-triol.

93. The method of Alternative 87, wherein the solid state form of 17α-ethynylandrost-5-ene-3β,7β,17β-triol is amorphous 17α-ethynylandrost-5-ene-3β,7β,17β-triol.

94. The method of any one of Alternatives 65 to 93, wherein the composition contains less than about 3% by weight of impurities.

95. A method to treat, prevent or decrease TNF production or TNF activation in a subject in need thereof, the method comprising: identifying a subject having a waist to hip ratio greater than approximately 0.80; and administering to the subject a therapeutically effective amount of a composition comprising 17α-ethynylandrost-5-ene-3β,7β,17β-triol and at least one pharmaceutically acceptable excipient, thereby treating, preventing or decreasing TNF production or TNF activation in the subject in need thereof.

96. The method of alternative 95, wherein administering to the subject 17α-ethynylandrost-5-ene-3β,7β,17β-triol decreases TNF production and TNF activation of a subjects TNFR1 receptor.

97. The method of alternative 95 or 96, wherein administering to the subject 17α-ethynylandrost-5-ene-3β,7,17β-triol decreases, prevents, or ameliorates metabolic inflammation.

98. The method of any one of alternatives 95 to 97, wherein administering to the subject 17α-ethynylandrost-5-ene-3β,7β,17β-triol prevents TNFR1 receptor phosphorylation in Ik/MAP3K8/MEK dependent scaffolds.

99. The method of any one of alternatives 95 to 98, wherein treating, preventing, or decreasing TNF production or TNF action is decreased by approximately 5% to about 99%.

100. The method of any one of alternatives 95 to 97, wherein the subject has a waist to hip ratio greater than approximately than 0.80 is female.

101. The method of alternative any one of alternatives 95 to 100, wherein the subject has a waist to hip ratio greater than approximately 0.95 is male other than Asian or Hispanic.

102. The method of alternative any one of alternatives 95 to 100, wherein the subject has a waist to hip ratio is greater than approximately 0.90 and the subject is Hispanic or Asian.

103. The method of alternative 95 to 102, wherein the subject experiences an improvement in overall cognitive function.

104. The method of alternative 103, wherein the subject experiences an improvement in overall cognitive function ranging from approximately 5% to 100% of their mid-life cognitive function or function before disease onset.

105. The method any one of alternatives 95 to 104, wherein the subject experiences an improvement in memory recall.

106. The method of alternative 105, wherein the subject experiences an improvement in memory recall ranging from approximately 5% to 100% of their mid-life cognitive function or function before disease onset.

107. The method any one of alternatives 95 to 106, wherein the subject experiences an improvement in memory loss.

108. The method of alternative 107, wherein the subject experiences an improvement in memory loss ranging from approximately 5% to 100% of their mid-life cognitive function or function before disease onset.

109. The method any one of alternatives 95 to 108, wherein the subject experiences an improvement in cloudy memory.

110. The method of alternative 109, wherein the subject experiences an improvement in cloudy memory ranging from approximately 5% to 100% of their mid-life cognitive function or function before disease onset.

111. The method any one of alternatives 95 to 110, wherein the subject experiences an improvement in mild cognitive dysfunction.

112. The method of alternative 111, wherein the subject experiences an improvement in mild cognitive dysfunction ranging from approximately 5% to 100% of their mid-life cognitive function or function before disease onset.

113. The method of any one of alternatives 95 to 112, wherein the subject have a mini mental state exam score ranging from approximately 12-29.

114. The method of any one of alternatives 95 to 113, wherein the 17α-ethynylandrost-5-ene-3β,7β,17β-triol is administered orally.

115. The method of any one of alternatives 95 to 113, wherein the 17α-ethynylandrost-5-ene-3β,7β,17β-triol is administered intravenously.

116. The method of any one of alternatives 95 to 115, wherein the subject has a waist to hip ratio greater than or equal to approximately 0.90.

117. The method of any one of alternatives 95 to 115, wherein the subject has a waist to hip ratio greater than or equal to approximately 0.95.

118. The method of any one of alternatives 95 to 117, wherein the 17α-ethynylandrost-5-ene-3β,7β,17β-triol is a solid state form of 17α-ethynylandrost-5-ene-3β,7β,17β-triol.

119. The method of Alternative 118, wherein the solid state form of 17α-ethynylandrost-5-ene-3β,7β,17β-triol is crystalline solvate of 17α-ethynylandrost-5-ene-3β,7β,17β-triol.

120. The method of Alternative 119, wherein the crystalline solvate is crystalline methanolate 17α-ethynylandrost-5-ene-3β,7β,17β-triol.

121. The method of Alternative 119, wherein the crystalline solvate is crystalline ethanolate 17α-ethynylandrost-5-ene-3β,7β,17β-triol.

122. The method of Alternative 119, wherein the crystalline solvate is crystalline hydrate 17α-ethynylandrost-5-ene-3β,7β,17β-triol.

123. The method of Alternative 119, wherein the crystalline solvate is Form III 17α-ethynylandrost-5-ene-3β,7β,17β-triol.

124. The method of Alternative 119, wherein the crystalline solvate is Form IV 17α-ethynylandrost-5-ene-3β,7β,17β-triol.

125. The method of Alternative 119, wherein the crystalline solvate is Form V 17α-ethynylandrost-5-ene-3β,7β,17β-triol.

126. The method of Alternative 118, wherein the solid state form of 17α-ethynylandrost-5-ene-3β,7β,17β-triol is amorphous 17α-ethynylandrost-5-ene-3β,7β,17β-triol.

127. The method of any one of Alternatives 95 to 126, wherein the composition contains less than about 3% by weight of impurities.

128. A method of treating, managing, ameliorating, or improving a psychological condition in a subject in need thereof, the method comprising: administering to the subject a therapeutically effective amount of a composition comprising 17α-ethynylandrost-5-ene-3β,7β,17β-triol and at least one pharmaceutically acceptable excipient, thereby treating, managing, ameliorating, or improving the psychological condition.

129. The method of alternative 128, wherein the psychological condition is selected from the group consisting of depression, anxiety, low self-esteem, low motivation, apathy, mental clarity, attention disorders, disorders of executive function, cognitive engagement, obsessive compulsive disorder, feelings of hope, feelings of independence, or combinations thereof.

130. The method of alternative 128 or 129, wherein treating, managing, ameliorating, or improving the psychological condition of the subject improves one or more of mood, self-esteem, mental clarity, feelings of hope, and feelings of independence.

131. The method of any one of alternatives 128 to 130, wherein treating, managing, ameliorating, or improving the psychological condition of the subject decreases one or more of depression, and apathy.

132. The method any one of alternatives 128 to 131, wherein the subject experiences an improvement in memory loss.

133. The method of alternative 132, wherein the subject experiences an improvement in memory loss ranging from approximately 5% to 75%.

134. The method of any one of alternatives 128 to 133, wherein the subject experiences an improvement in cloudy memory.

135. The method of alternative 134, wherein the subject experiences an improvement in cloudy memory ranging from approximately 5% to 75%.

136. The method of any one of alternatives 128 to 135, wherein the subject experiences an improvement in mild cognitive dysfunction.

137. The method of alternative 136, wherein the subject experiences an improvement in mild or moderate cognitive dysfunction ranging from approximately 5% to 75%.

138. The method of any one of alternatives 128 to 137, wherein the subject has a mini mental state exam score ranging from approximately 12-29.

139. The method of any one of alternatives 128 to 138, wherein the subject experiences an improvement in overall the psychological condition ranging from approximately 5% to 75%.

140. The method of any one of alternatives 128 to 138, wherein the subject experiences a decrease in overall the psychological condition ranging from approximately 5% to 75%.

141. A method of treating, managing, ameliorating, or improving a behavioral condition in a subject in need thereof, the method comprising: administering to the subject 17α-ethynylandrost-5-ene-3β,7β,17β-triol and at least one pharmaceutically acceptable excipient, thereby treating, managing, ameliorating, or improving the behavioral condition.

142. The method of alternative 141, wherein the behavioral condition is selected from the group consisting of self-care, agitation, aggression, restlessness, wandering, physical outbursts, verbal outbursts, pacing, irritability, engagement, or combinations thereof.

143. The method of alternative 141 or 142, wherein treating, managing, ameliorating, or improving the behavioral condition of the subject improves one or more of self-care, agitation, aggression, restlessness, and engagement.

144. The method of any one of alternatives 141 or 143, wherein treating, managing, ameliorating, or improving the behavioral condition of the subject decreases one or more of agitation, aggression, restlessness, wandering, physical outbursts, verbal outbursts, pacing, and irritability.

145. The method of any one of alternatives 141 or 144, wherein the subject experiences an improvement in overall the behavioral condition ranging from approximately 5% to 75%.

146. The method of any one of alternatives 141 or 144, wherein the subject experiences a decrease in overall the behavioral condition ranging from approximately 5% to 75%.

147. A method of treating, managing, ameliorating, or improving a physical condition in a subject in need thereof, the method comprising: administering to the subject 17α-ethynylandrost-5-ene-3β,7β,17β-triol and at least one pharmaceutically acceptable excipient, thereby treating, managing, ameliorating, or improving the physical condition.

148. The method of alternative 147, wherein the physical condition is selected from the group consisting of weight loss, loss of motor coordination and balance, muscle weakness and stiffness, pain, chronic pain, headaches, sleep, movement, fatigue, sleep disturbance, eating disorders, or combinations thereof.

149. The method of alternative 147 or 148, wherein treating, managing, ameliorating, or improving the physical condition of the subject improves one or more of weight, motor coordination and balance, muscle weakness and stiffness, energy, sleep, movement, and fatigue.

150. The method of any one of alternatives 147 or 149, wherein treating, managing, ameliorating, or improving the physical condition decreases one or more of loss of weight, loss of balance, muscle weakness and stiffness, pain, chronic pain, headaches, sleep disturbances, and eating disorders.

151. The method of any one of alternatives 147 to 150, wherein the subject experiences an improvement in overall the physical condition ranging from approximately 5% to 75%.

152. The method of any one of alternatives 147 to 150, wherein the subject experiences a decrease in overall the physical condition ranging from approximately 5% to 75%.

153. The method of any one of alternatives 128 to 150, wherein the subject has a neurodegenerative condition.

154. The method of alternative 153, wherein the neurodegenerative condition is mild or moderate cognitive impairment.

155. The method of alternative 154, wherein the mild or moderate cognitive impairment is early onset Alzheimer's disease or probable Alzheimer's disease.

156. The method of alternative 154, wherein the neurodegenerative condition or disease is dementia.

157. The method of alternative 156, wherein the subject has a clinical dementia rating ranging from approximately 0.5-2.

158. The method of any one of alternatives 128 to 157, wherein the 17α-ethynylandrost-5-ene-3β,7β,17β-triol is administered orally.

159. The method of any one of alternatives 128 to 157, wherein the 17α-ethynylandrost-5-ene-3β,7β,17β-triol is administered intravenously.

160. The method of any one of alternatives 128 to 159, wherein the 17α-ethynylandrost-5-ene-3β,7β,17β-triol is a solid state form of 17α-ethynylandrost-5-ene-3β,7β,17β-triol.

161. The method of alternative 160, wherein the solid state form of 17α-ethynylandrost-5-ene-3β,7β,17β-triol is crystalline solvate of 17α-ethynylandrost-5-ene-3β,7β,17β-triol.

162. The method of alternative 161, wherein the crystalline solvate is crystalline methanolate 17α-ethynylandrost-5-ene-3β,7β,17β-triol.

163. The method of alternative 161, wherein the crystalline solvate is crystalline ethanolate 17α-ethynylandrost-5-ene-3β,7β,17β-triol.

164. The method of alternative 161, wherein the crystalline solvate is crystalline hydrate 17α-ethynylandrost-5-ene-3β,7β,17β-triol.

165. The method of alternative 161, wherein the crystalline solvate is Form III 17α-ethynylandrost-5-ene-3β,7β,17β-triol.

166. The method of alternative 161, wherein the crystalline solvate is Form IV 17α-ethynylandrost-5-ene-3β,7β,17β-triol.

167. The method of alternative 161, wherein the crystalline solvate is Form V 17α-ethynylandrost-5-ene-3β,7β,17β-triol.

168. The method of alternative 160, wherein the solid state form of 17α-ethynylandrost-5-ene-3β,7β,17β-triol is amorphous 17α-ethynylandrost-5-ene-3β,7β,17β-triol.

169. The method of any one of alternatives 128 to 168, wherein the composition contains less than about 3% by weight of impurities.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

Evidence of probable metabolic inflammation, which scientific evidence supports as fundamental to AD, can be measured in vulnerable populations through common laboratory tests of metabolic function such as fasting plasma glucose and insulin and the temporal instability of these laboratory parameters relative to non-AD vulnerable populations. Recent medical evidence as also characterized visceral adipose tissue as an important source of inflammation that promotes neuroinflammation in AD vulnerable populations. Although such central adiposity may be readily observed in the AD vulnerable in mid-life and measured as high body mass index (BMI), body composition changes associated with aging, especially the loss of muscle mass, can decrease body weight and BMI, even while retaining central adiposity. Certain of these vulnerable individuals will have converted from high BMI in mid-life to high waist to hip ratio (WHR) later in life, with their central fat mass fueling a systemic inflammatory state that can slowly feed forward to promote neurodegenerative disease. Several longitudinal studies have followed inflammatory disease vulnerability in high WHR populations, which have led to anthropomorphic characterizations describing risk, which vary by gender and ethnicity. Thus individuals with high WHR for their gender and ethnicity and evidence of cognitive decline beyond the expected norm for their age have an extremely elevated risk of developing AD related dementias and could be logical candidates for AD therapy if the relationship between WHR and response to a therapy was known to apply for amyloid/p-tau diagnostically positive and negative populations. WHR boundaries were derived from patients with metabolic disease, but the relationship of metabolic disease to AD provides a rationale for the use of these published values for AD: Caucasian female >0.8, Caucasian male >0.95, Asian male >0.90. However, the broad implementation of the amyloid hypothesis disease diagnosis demands AD pathology specific diagnostic tests involving amyloid beta or p-tau, and the aforementioned WHR relationship to amyloid/p-tau is not known publicly. Anti-inflammatory therapy in the CNS is hampered by the lack of safe medications with mechanisms of action that can effectively dampen the complex inflammatory environment that exists in the AD brain. All currently approved anti-inflammatory therapies do not discriminate between homeostatic and pathological activities of inflammatory mediators, which makes their chronic use generally unacceptable for AD. (It is important to note that all inflammatory mediators also have homeostatic functions. For example, tumor necrosis factor, well known as a critical mediator of AD pathology, has important homeostatic roles in astrocyte and GABA interneurons in the hippocampus, which decreases the safety of non-selective anti-inflammatory therapies.) Apparent pathology specific anti-inflammatory activity has been achieved with 17α-ethynylandrost-5-ene-3β,7β,17β-triol in various disease models. Clinical trials in AD and Parkinson's disease (PD) indicate that 17α-ethynylandrost-5-ene-3β,7β,17β-triol also has pathology specific anti-inflammatory activity against neuroinflammatory disease in humans.

In a trial, the relationship between Clinical Dementia Rating Scale (CDR) and Mini Mental State Exam (MMSE) scores with measures of obesity, including Body Mass Index (BMI), Waist Circumference (WC), and waist-to-hip ratio (W/H) was explored with administration of 17α-ethynylandrost-5-ene-3β,7β,17β-triol. The trial sought to explore the anti-inflammatory and insulin sensitizing activity of 17α-ethynylandrost-5-ene-3β,7β,17β-triol in subjects with dementia, using inclusion criteria of the CDR levels 1-2 and the MMSE scores of 14-24 for inclusion criteria.

Alzheimer's Disease and Related Dementias (ADRD) are also well known to be associated with systemic and neuroinflammation, and the clinical diagnosis of Alzheimer's Disease (AD) is based on cognitive and memory decline, and the inability of a subject to care for oneself. APTUS-Aβ blood tests, referred to as APTUS or Aptus tests, are in vitro diagnostic tests designed to measure amyloid-beta proteins from blood samples, which can be used to predict amyloid PET scan results. Clinical trials screen subjects using the CDR score and the MMSE score for inclusion, and if an amyloid therapy is being trialed, additional measures for amyloid by positron emission tomography (PET) of APTUS may be added as inclusion criteria, since testing an amyloid therapy in a subject without increased amyloid would not make sense.

In this trial, the correlation between CDR (1-2) and MMSE (14-24) scores and the presence or absence of amyloid by the Aptus test, and also the presence of absence of metabolic inflammation using markers of obesity and metabolic inflammation was explored.

In attempts to measure amyloid in the brain of live subjects, PET using radioactive ligands that bind to amyloid beta plaques have been used for detection of “plaque-entangled disease”. The imaging results were correlated with post-mortem plaques. It was not a one-to-one relationship. Due to the great expense and the use of radioactivity for PET, further correlations have been found between the ratios of two fragments of amyloid beta in the blood (Ab42/Ab40 ratio) and PET scans for amyloid plaques. This correlation with a correlation approach has led to a ratio that cannot stand alone, but is dependent on the association with age, gender and apolipoprotein E (APOE) genotype. This complex analysis “Aptus Test” has been used to associate the presence of amyloid fragments in the blood with PET evidence of amyloid plaques in the brain, which is further correlated with post-mortem plaques in AD subjects.

With Aptus data available for 96 subjects that met the criteria for CDR and MMSE, only 35 subjects (36%) met the Aptus criteria. When measures of obesity were examined, BMI was available for 149 subjects. Due to the age-related decrease in muscle mass, only 60 (40%) of the subjects met the CDC criteria (male BMI >40, Female BMI >35) for obesity. These restrictions would severely limit enrollment. Analysis of the waist circumference was somewhat better, as the central adiposity is known to be associated with metabolic inflammation, yielding 98 of 149 subjects (66%) with WC measurements according to the CDC (Male WC >40; Female WC >35). We then examined the waist-to-hip ratio (W/H) and from 148 subjects with these measurements, 131 (89%) met the HHS criteria.

Based on these findings, protocols have been modified to enroll both Aptus positive and Aptus negative subjects. The results showed improvements in cognition independent of amyloid beta positivity. The waist to hip ratio but not BMI, correlated closely with cognitive impairment consistent with probable AD that could be used with MMSE and CDR ratings to determine inclusion in dementia and Alzheimer's studies. These indicators show a link between inflammation and visceral adiposity with neuroinflammatory disease and cognitive impairment such as dementia observed in probable Alzheimer's disease and related dementias.

### Example 2

Twenty-three patients, approximately equal numbers of men women, age 50-89 years, with cognitive impairment consistent with MCI or mild probable AD, but without other readily apparent major systemic disease were identified and received informed consent under the supervision of a central institution review board (IRB). Patients were qualified (screened) for the study by the following criteria, QDRS score ranging from 1.5-12.5, with a converted Clinical Dementia Rating (CDR) score of 0.5 (mild cognitive impairment) to 1 (mild dementia).

Prior to treatment (baseline) concomitant medications were stabilized and the patients were tested for cognitive function by the Quick Dementia Rating Scale (QDRS), Clinical Dementia Rating (CDR) score as estimated by the QDRS, a modified form of the Alzheimer's Disease Assessment Scale (ADAS-Cog12), the Mini-Mental State Examination (MMSE), and the Montreal Cognitive Assessment (MoCA) and by multi-modal brain MRIs to establish pre-treatment data for glutathione levels (as measured by magnetic resonance spectroscopy, MRS), dendritic density (as measured by diffusion tensor imaging, DTI-NODDI); arterial signal compared (as quantified by arterial spin labeling, ASL), functional connectivity of the nucleus basalis of Meynert (NBM) with both hippocampi as well as between both hippocampi as visualized by seed analysis of blood-oxygen level dependent (BOLD) imaging, and neurovascular coupling as visualized by BOLD compared to baseline. Baseline samples of the patients' blood and cerebral spinal fluid were collected for evaluation of AD and inflammation biomarkers.

Patients were treated with 20 mg 17α-ethynylandrost-5-ene-3β,7β,17β-triol oral capsules twice daily (BID for three months. All patients received 17α-ethynylandrost-5-ene-3β,7β,17β-triol; there were no placebo controls. Safety was monitored at clinical visits at 2 weeks, 4 weeks, 8 weeks, and completion. Routine hematology and clinical chemistry tests were performed at these visits and adverse events were monitored.

At the end of the treatment schedule the baseline assessments for cognition, neuroimaging and biomarkers were repeated.

### Results

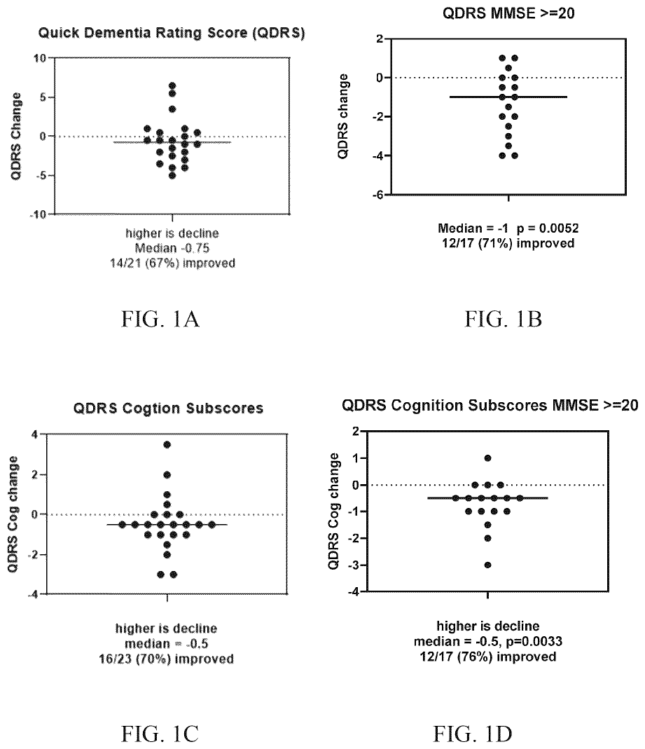

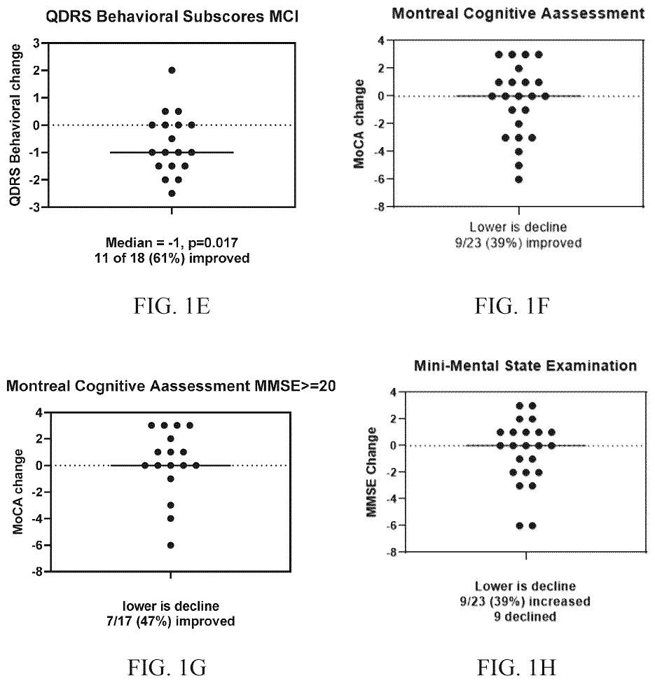

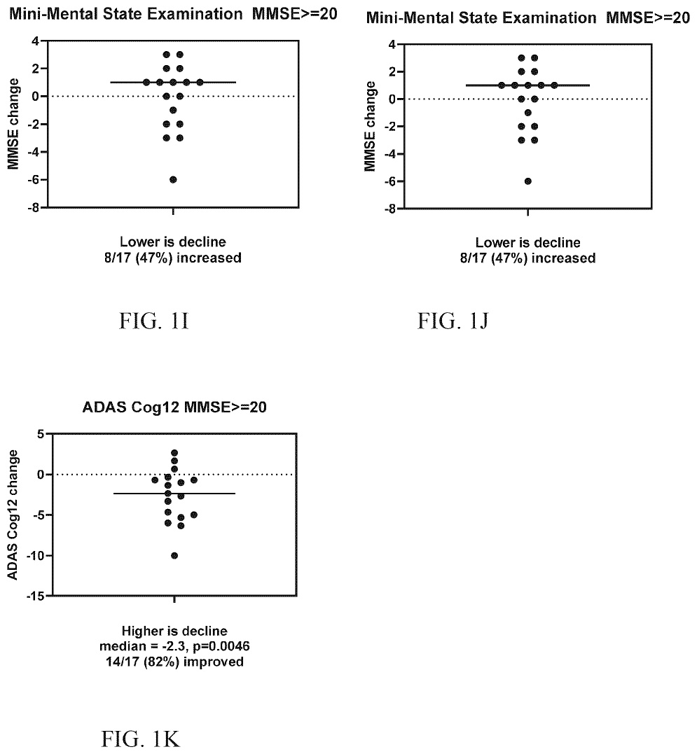

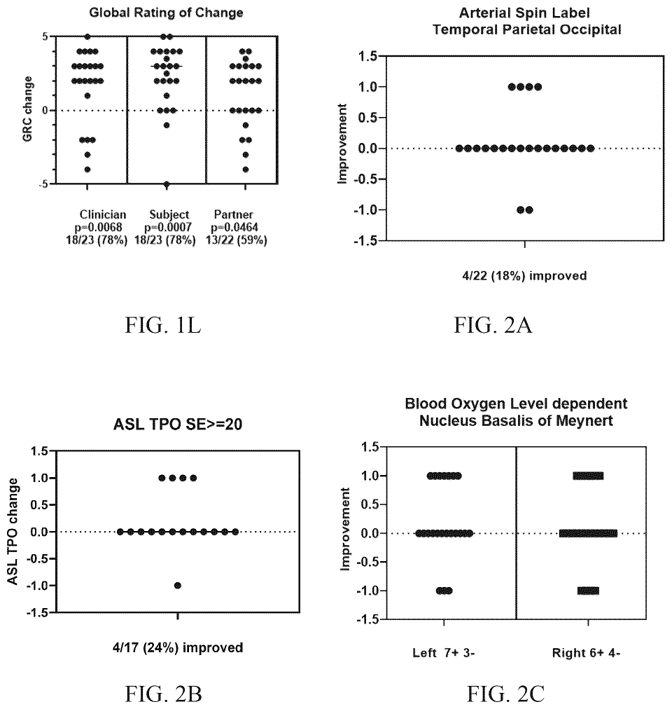

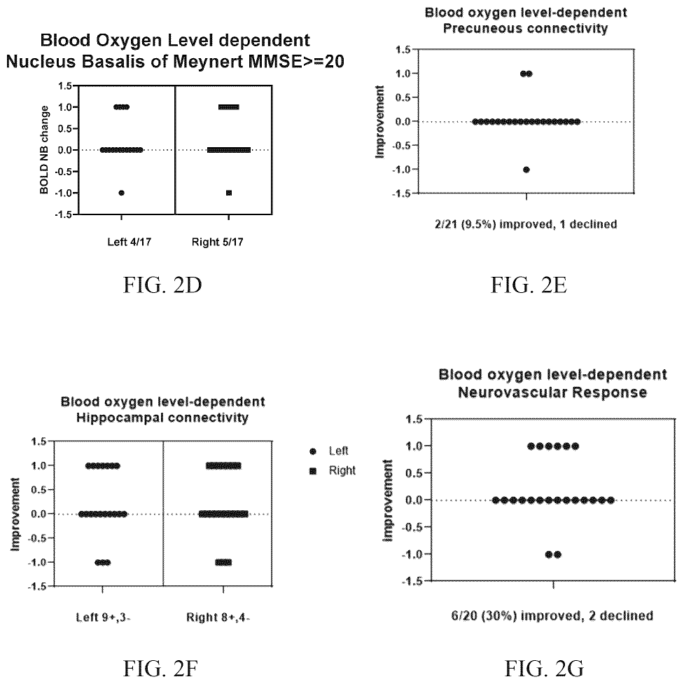

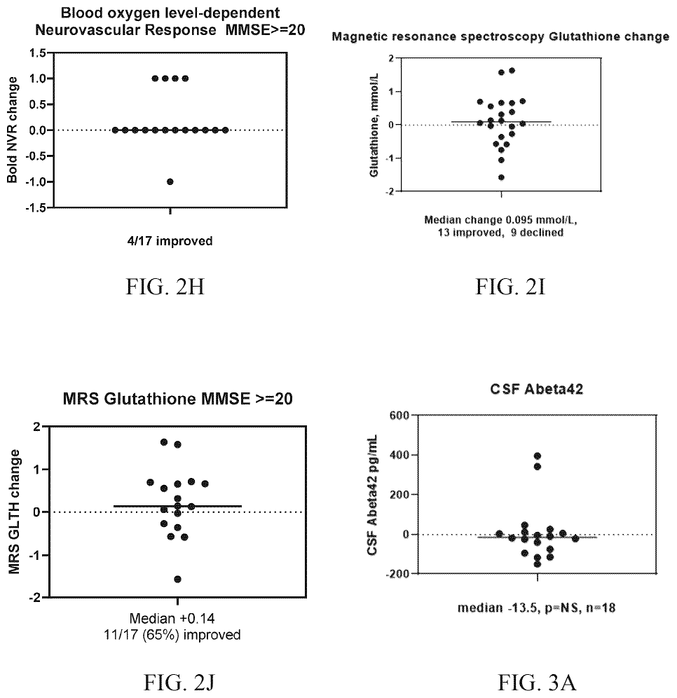

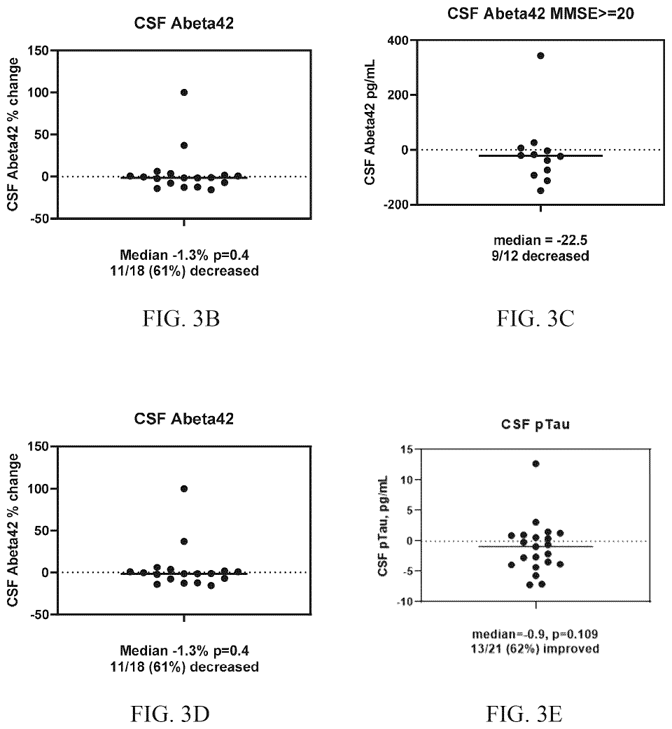

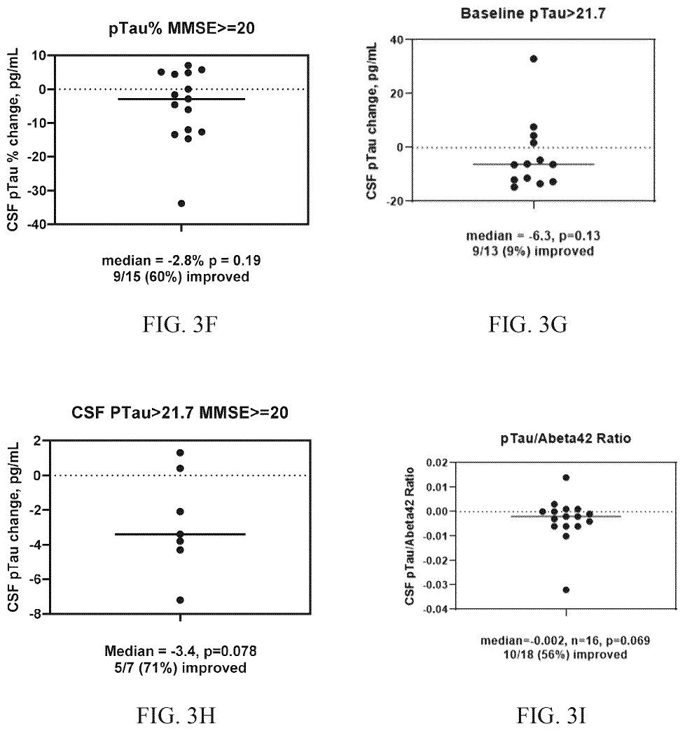

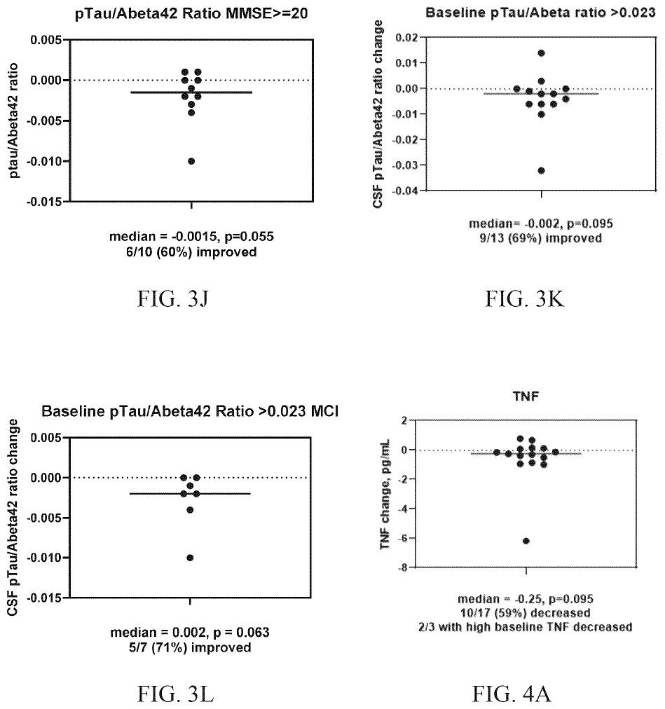

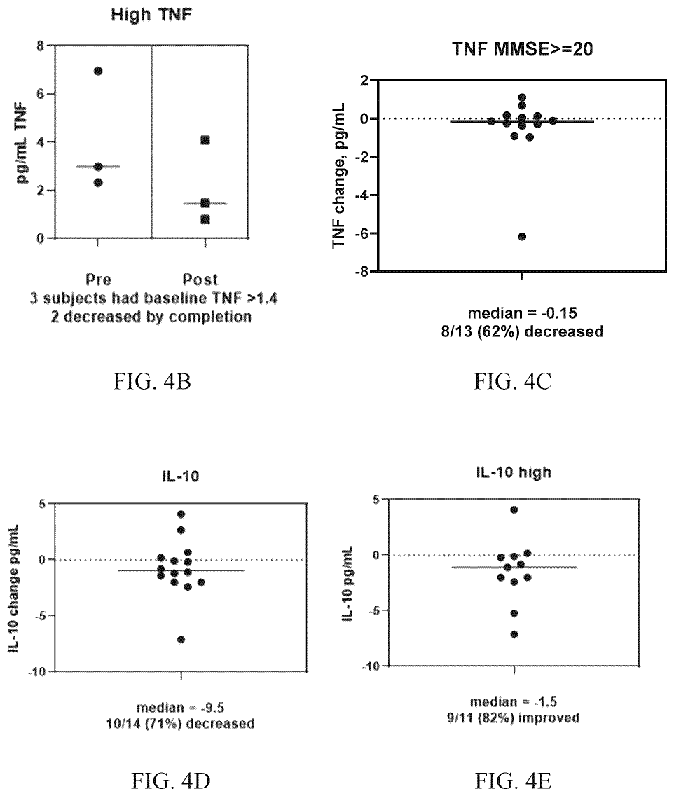

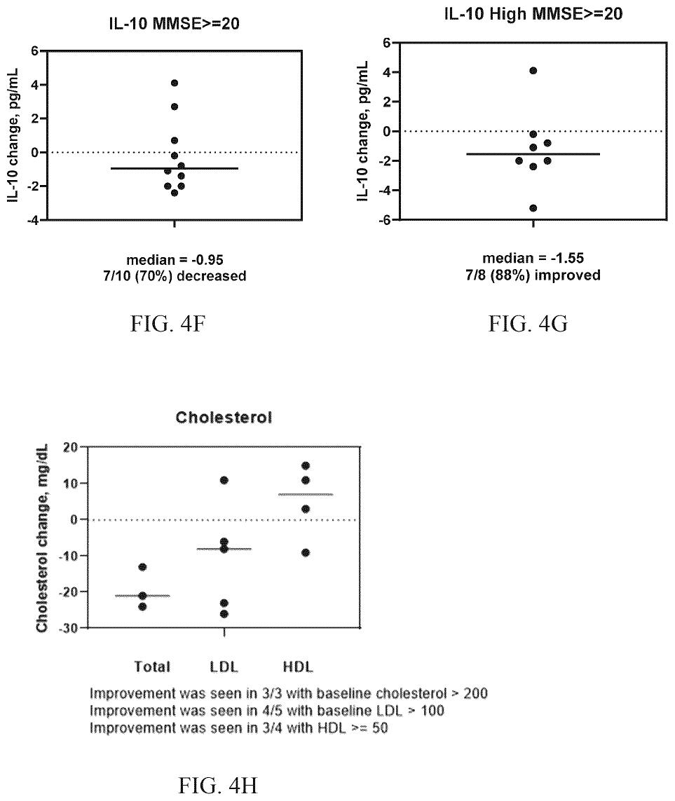

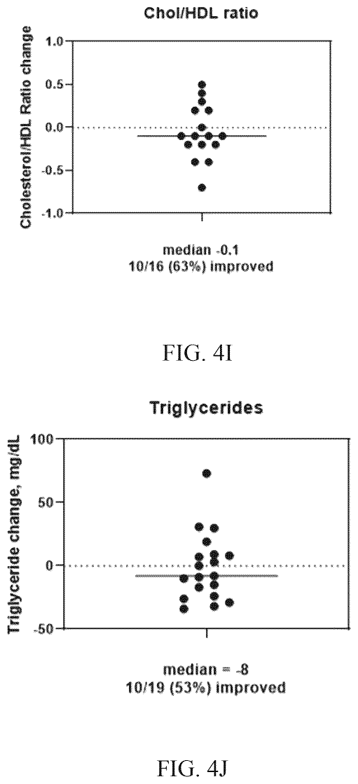

17α-ethynylandrost-5-ene-3β,7β,17β-triol decreased the QDRS, with a median decrease of 0.75 points; 14 of 21 patients evaluated improved. The median score for patients with a baseline MMSE score 20 improved one point; 12/17 improved. Median QDRS cognition subscores improved (declined) by 0.5 points, 16/23 patients improved. Median QDRS behavioral for MCI patients improved one point, 11 of 18 improved. In the overall population, 9 of 23 patients improved Montreal Cognitive Assessment. For MCI patients, 7 of 17 improved with the Montreal Cognitive Assessment. In the overall population, 9 of 23 improved the MMSE score. In the MCI population, 8 of 17 improved the MMSE score. In the overall population, 14 of 23 improved the ADAS Cog 12 score; the median score improved 0.67 points. In the MCI population, 14 of 17 improved the ADAS Cog 12 score; the median score improved 2.3 points. In the Global Rating of Change (GRS) that is evaluated by the clinician, by the patient, and by the patient's study partner, the clinician's score improved in 18 of 23, the patient's self-evaluation score also improved in 18 of 23, and the score from study partners improved in 13 of 22. GSR scores for MCI patients improved 16 of 17 by the clinician, 15 of 17 by patients, and 11 of 17 by study partners. Results are summarized in FIG. 1A-1L. The results of the neuroimaging study are summarized in FIG. 2A-2J. The results of CSF biomarker analysis are summarized in FIG. 3A-3L. The results of peripheral blood biomarker analysis are summarized in FIG. 4A-4J.

### Example 3

An exploratory Phase 2 trial of 17α-ethynylandrost-5-ene-3β,7β,17β-triol (NE3107-TRP-001) in 23 patients with mild (n=17) or moderate dementia (n=6) were conducted. Comparisons between baseline and post-intervention (3 months) were evaluated via multi-modal brain magnetic resonance images (MRIs; primary endpoints), measures of glucose and insulin homeostasis, and neuropsychological testing (secondary endpoints). Most patients with mild dementia showed significant improvements in cognition, including a 2.6-point decrease (p=0.0046) on the Alzheimer's Disease Assessment Scale-Cognitive Subscale 11 (ADAS-Cog 11), and improvements on the Global Rating of Change (GRC).

Given its novel mechanism of action, qualitative data were collected via exit interviews with a subset of patients (and study partners, if applicable) who completed NE3107-TRP-001 in order to identify meaningful changes according to patients/study partners that will be incorporated into the NE3107 development program for degenerative dementia. These data may also potentially inform early advisory interactions with regulatory and health technology assessment (HTA) bodies, in both United States (US) and ex-US settings.

The objectives of these exit interviews were to characterize the patient/study partner experience with NE3107, including changes in degenerative dementia symptoms, unexpected benefits, and whether the changes were meaningful to them.

The exit interviews were conducted in the US with 11 adult patients/study partners who completed the Phase 2 trial of NE3107. Participants (patients and study partners) completed one 60-minute semi-structured interview via remote videoconference to facilitate face-to-face communication. The US Food and Drug Administration (US FDA) has recently noted that current evidence does not suggest any substantial difference between modes of interview (video, telephone, in-person) with respect to the accuracy of collected information (US FDA 2022), which is supported by studies examining the comparability between interview modes (Krouwel et al. 2019; Macey et al. 2019).

Each interview was divided into four parts: 1) elicitation of experience with degenerative dementia, 2) changes due to treatment with NE3107 and meaningfulness of those changes, 3) experience with NE3107 during the trial, and, if applicable, 4) study partner perceptions of the patient's changes during the trial and their trial experience.

Prior to study implementation, the semi-structured interview guide was piloted with two internal team members as mock interviewees. The goals of the pilot interviews were to: 1) gauge the length of the interview with the guide as-written, 2) identify areas to reduce or expand probing depending on length, and 3) identify any need for restructuring of questions to streamline interview flow. Following the mock interviews, no significant changes were needed.

Interviews took place via videoconference with a trained and experienced interviewers using a semi-structured interview guide. Participants were required to confirm that they signed the ICF for the exit interview, and provide permission to record the interview. The interviews lasted approximately 60 minutes. Open-ended questions were used to characterize the participant's experience with degenerative dementia. Three overarching topics were covered: 1) degenerative dementia signs and symptoms prior to the trial, 2) changes experienced during the trial, and 3) experience with the trial medication (NE3107). Interviewers noted spontaneously reported changes experienced during the trial and followed up with probes (as needed) to elicit the full range of changes experienced.

If both the patient and study partner participated, the patient was interviewed first with the study partner present. Following the patient interview, the study partner was interviewed without the patient present. Interviewers were instructed to direct the questions towards the patient and to remind the study partner that they would have an opportunity to add information or different perspectives at the end.

The coding and analysis of the interview transcripts followed principles in line with qualitative thematic (content) analysis (Braun et al. 2019), with additional features drawn from grounded theory (Bryant and Charmaz 2007; Strauss and Corbin 1998). This approach conforms to best practices in the field (Patrick et al. 2011). A saturation table was developed during the coding process and updated after each interview to ensure that the interviews provided sufficient quality and quantity of data to support the study goals. The focus of saturation was changes due to treatment.

Quality assurance and quality control systems were implemented in accordance with standard operating procedures to ensure that the interviewers were conducted, documented, and reported in compliance with the study protocol and applicable regulatory requirements.

The quality of the interviews was monitored regularly and ensured by the qualifications and experience of the interviewers. Study-specific training was conducted to orient the interviewers to the purpose of the study and to the semi-structured interview guide. Throughout the interview period, the interview recordings were reviewed for compliance with interview administration; no major deviations were noted. The quality of the coding was ensured by the use of reconciliation for the first three interview transcripts. Continued oversight was performed during coding and analysis. Reconciliation rules were defined and recorded for subsequent coding activities.

### Results

A total of 24 patients participated in the NE3107 trial, 13 (54.2%) of whom did not participate in exit interviews. Twelve informed site staff they were not interested in the exit interview and one patient signed the exit interview ICF but then withdrew via informing clinical site staff. Of the 11 patients interviewed, the average age was 70 years, most were female (n=9; 81.8%), and all were White/Non-Hispanic (Table 1). Very few presented with comorbidities (for example, stroke, concussion, etc.), and roughly one-third had a family history of either AD (n=4; 36.4%) or dementia (n=3; 27.3%). Seven (63.6%) patients completed interviews with a study partner and the remaining four (36.4%) participated independently.

TABLE 1

| | |
|---|---|
| Characteristic | Statistic |

Demographic

| | |
|---|---|
| Age (years), mean (SD) [range] | 70.4 (6.32) [60,81] |
| Sex, n (%) | |
| Female | 9 (81.8) |
| Male | 2 (18.2) |
| Ethnicity, n (%) | |
| Non-Hispanic | 11 (100.0) |
| Race, n (%) | |
| White | 11 (100.0) |

Clinical

| | |
|---|---|
| Traumatic Brain Injury, n (%) | 1 (9.1) |
| Concussion, n (%) | 2 (18.2) |
| Stroke, n (%) | 1 (9.1) |
| High Blood Pressure, n (%) | 2 (18.2) |
| High Cholesterol, n (%) | 2 (18.2) |
| Sleep Apnea, n (%) | 2 (18.2) |
| Major Depressive Disorder n (%) | 2 (18.2) |
| Family History, n (%)¹ | |
| Alzheimer's Disorder | 4 (36.4) |
| Dementia, unspecified Etiology | 3 (27.3) |

¹Not mutually exclusive

Results from the interviews are organized by participant type (patient or study partner) and topic. Representative verbatim quotes are presented as italicized text and identified by their study ID number.

### Patients

At the beginning of the interview, patients were asked open-ended questions about their experience with degenerative dementia prior to enrolling in the NE3107 trial. A total of 14 unique concepts related to their experience were reported and categorized as follows: cognitive (2 concepts), physical (5 concepts), psychological (2 concept), behavioral (2 concepts) and impact on overall functioning (3 concepts) (Table 2).

All patients reported at least one cognitive problem and most reported at least one physical problem (n=7; 63.6%), psychological problem (n=7; 63.8%) and impact on overall functioning (n=9; 73.7%). Patients reported an average of 4.6 (SD=2.02, range 1-7) problems and impacts related to their degenerative dementia.

The most commonly reported issues were cognitive in nature, specifically difficulties with memory or clarity.

### Memory

All patients (n=11; 100.0%) described memory difficulties such as forgetting names, people, places, and things, as well as word-finding and brain freezes.

“I had issues with names. I'd have to think about a name in particular for a while in order to remember that name if it had been awhile since I had met that person.” [N-18-22]

“I pretty much call it a brain freeze, you know, the instant forgetting, just like why did I walk there or what was I going to pick up or just stuff like that . . . it was just happening a lot and like I say, it just seemed atypical from the usual forgetting.” [N-03-22]

“ . . . like first started to forget appointments and yeah, it just got too crazy after a while, and then losing things and misplacing things. And it got to the point where I'd have a checkbook one minute and then it'd take four hours to find where I put it. Or I put things in a place that I just said okay, remember this is where you're putting it and you'll never forget. Took me three days to find out where it was. And then it's just getting more and more and more like that.” [N-17-22]

“Well, it's just like struggle finding the last word and hesitating before I finish a thought . . . ” [N-02-22]

### Clarity

Issues with clarity or thinking clearly were noted by a majority of patients (n=7; 63.6%). Clarity encompassed difficulty understanding others, problems with organizing thoughts or processing information, focus, decision-making, and orientation.

“ . . . I went back east to see my sister for a week and then when I came back they changed the lift at the LAX and you have to take like a shuttle to the place . . . I was calling all these numbers and no one—and it was like I was like I was there three hours . . . And I just—I couldn't—for whatever reason—I mean we were 10 miles away. Like we're not 100 miles. You know, I couldn't figure out. You know now I know—when I thought about it days later I was like why didn't I just go to like the taxicab stand or why didn't—I don't know. I found myself I was walking with my huge luggage and everything down this like dark street and then I went back because this cop was like where are you going. I was like I don't know [laughs], you know. So that was kind of—that was a little weird. And I was upset that I wasn't able to coordinate that which normally I would, you know, like a new situation.” [N-02-22]

“If I went to certain places I'd be able to get there. Meaning that the more frequently I went to a place I'd be able to get there. But there were times that I mean I'd get lost. I mean I would get lost. So I usually try to go to familiar places.” [N-07-22]

“ . . . one thing that I noticed is I used to love to read books. And I just couldn't even do it anymore so I started getting books on tape and doing it that way. So that was a big change as well.” [N-17-22]

Several patients (n=6; 54.5%) reported issues with mood (psychological) typically related to frustration and depression.

### Mood

“Frustration, just a lot of frustration. Like I say, I could go, rewind slowly, and usually get back to whatever I was attempting to do, but still, just stopping me in my tracks, you know, throughout the day very frustrating.” [N-03-22]

“ . . . I felt sad and I felt perturbed because I couldn't get a word or I couldn't think of what I was trying to say, and I was looking for it in my brain, but I couldn't find it, so I was quite upset.” [N-16-22]

“I get up and have my coffee and not wake up and I'm just like lay back in bed and watch TV and hope the next day would be different.” [N-02-22]

Regarding physical issues, roughly half of the patients (n=5; 45.5%) noted a lack of energy or being tired, and three (27.3%) reported issues with movement/balance. Headaches, neuropathy, and numbness (all n=1; 7.7%) were less frequently reported.

### Energy

“Less energetic. And I know they were always asking if I'm—are you depressed, are you sad, and it wasn't anything like that because I know the difference. It was more of a slowing. But it was even more—like kind of more subdued.” [N-10-22]

“Well, I continued to get more tired, I was more frustrated and then I realized I had to make a different plan, because this one wasn't working. So, I had to input different things that I do in different ways, and I did take naps, and I'm not a nap person—it was exhausting.” [N-16-22]

### Movement/Balance

“Yeah, impaired balance. Like five years ago I could ride a bike, two years ago I couldn't. I didn't have good enough balance that I could trust to ride a bike.” [N-10-22]

“Sometimes it's my gait, walking. And my gait's always been pretty good.” [N-18-22]

Most patients (n=8; 72.7%) spoke of how their memory and thinking problems negatively impacted their day to day life prior to starting the NE3107 trial. Five patients (45.5%) described problems in their relationships and one patient (9.1%) noted difficulty with work.

Difficulty with Relationships

“When this came about, I started not interacting with people, because I felt like I was stupid because I couldn't find the things in my head. I didn't want to be that oh, my gosh, we're going out and it isn't fun, because [name 21:00] can't do this or that or remember that or talk about it or blah, yeah.” [N-16-22]

“[did your memory and thinking problems affect your ability to participate in social activities and get out with friends?] Yeah, yeah, for sure. You know, because not being able to remember names and then not even having the concentration or the motivation to do it.” [N-02-22]

Difficulties with engagement (behavioral), such as problems communicating with others and doing things they enjoy (including participation in solo activities), were discussed by three patients (27.3%). Two patients (18.2%) mentioned difficulty with self-care (behavioral).

### Engagement

“My biggest problem is just getting my thoughts out of my head, you know, just articulating. And that's frustrating to me sometimes.” [N-20-22]

“I used to be a real bookworm. You know, I'd want to read, want—and over the last few years if I can get through one in two months that's like lucky. So I know that that's a big—that's been a huge change for the last few years and I really—that like bothers me.” [N-02-22]

### Self-Care

“Not able to want to eat or fix food to eat, to not go anywhere, to not put anything but pajamas on, not dressing—that's all I've got.” [N-16-22]

Less frequently (n=2; 18.2%), patients cited a need to depend on others (impact) or being worried about their future (psychological).

### Depend on Others

“I have a family who helps, which is exactly what I need, but I can't stand the thought that I have to depend on them. Let's say we switch with who does the meals and who goes to the grocery and blah, blah, blah. I do not care for that situation.” [N-16-22]

Worry about Future

“Just envisioning the worst, really. I mean I saw my mother, I saw my grandmother, I saw my aunts, so I knew what Alzheimer's looked like, so that was scary.” [N-03-22]

TABLE 2
Pre-Trial Symptoms and Impacts (Patients; N = 11)

| | | | | | | | | | | | | Total | |
|---|---|---|---|---|---|---|---|---|---|---|---|---|---|
| ID | 03 | 16 | 07 | 18 | 10 | 02 | 14 | 17 | 20 | 22 | 23 | (n %) | |
| Cognitive | | | | | | | | | | | | | |
| Difficulty with | X | X | X | X | X | X | X | X | X | X | X | 11 | (100.0) |
| Memory | | | | | | | | | | | | | |
| Difficult with | X | | X | X | X | X | | X | | X | | 7 | (63.6) |
| Clarity | | | | | | | | | | | | | |
| Psychological | | | | | | | | | | | | | |
| Difficulty | X | X | | X | | | | X | | X | X | 6 | (54.5) |
| with Mood | | | | | | | | | | | | | |
| Fear or Worry | X | | | | | X | | | | | | 2 | (18.2) |
| of Future | | | | | | | | | | | | | |
| Physical | | | | | | | | | | | | | |
| Difficulty with | | X | | | X | X | | X | X | | | 5 | (45.5) |
| Energy | | | | | | | | | | | | | |
| Difficulty with | | | | X | X | | | | X | | | 3 | (27.3) |
| Movement | | | | | | | | | | | | | |
| Difficulty with | | | | | | | | | | | X | 1 | (9.1) |
| Headaches | | | | | | | | | | | | | |
| Neuropathy | | | | | X | | | | | | | 1 | (9.1) |
| Numbness | | | | | X | | | | | | | 1 | (9.1) |
| Impacts | | | | | | | | | | | | | |
| Difficulty with | X | X | | X | | X | | X | | | | 5 | (45.5) |
| Relationships | | | | | | | | | | | | | |
| Dependence | | X | | | | | | X | | | | 2 | (18.2) |
| on Others | | | | | | | | | | | | | |
| Difficulty | | | | | X | | | | | | | 1 | (9.1) |
| with Work | | | | | | | | | | | | | |
| Behavioral | | | | | | | | | | | | | |
| Difficulty with | | X | | | | | | | | X | X | 3 | (27.3) |
| Engagement | | | | | | | | | | | | | |
| Difficulty with | | X | | | | | | | X | | | 2 | (18.2) |
| Self-Care | | | | | | | | | | | | | |

| | | | | | | | | | | | |
|---|---|---|---|---|---|---|---|---|---|---|---|
| # of New | 5 | 4 | 0 | 1 | 3 | 0 | 0 | 0 | 0 | 0 | 1 |
| Concepts | | | | | | | | | | | |
| % of New | 35.7 | 28.6 | 0.0 | 7.1 | 21.4 | 0.0 | 0.0 | 0.0 | 0.0 | 0.0 | 7.1 |
| Concepts | | | | | | | | | | | |
| Cumulative % | 35.7 | 64.3 | 64.3 | 71.4 | 92.8 | 92.8 | 92.8 | 92.8 | 92.8 | 92.8 | 100.0 |
| of New Concepts | | | | | | | | | | | |
| Total Concepts | 5 | 7 | 2 | 5 | 7 | 5 | 1 | 7 | 4 | 4 | 3 |
| per Interview | | | | | | | | | | | |

Bold = first appearance of concept

Before participating in the NE3107 trial, the majority of patients (n=7; 63.6%) found memory difficulties to be the most challenging. Others described issues with thinking clearly (n=3; 27.1%) and mood (n=2; 18.2%) as most challenging.

“Remember things, all the—yeah, remembering things that I was supposed to do in the daytime and if I didn't write it down remembering when I had appointments, like doctors' appointments, returning phone calls that I was supposed to . . . ” [N-17-22]

“For me, honestly it was just not being able to remember things. It's mostly names and numbers. Math scares the willies out of me because I know that if you ask me something I won't be able to tell you right on the spot. I don't have confidence in my spelling anymore, because—I don't know why.” [N-20-22]

During the interviews, patients were asked open-ended questions about any changes they experienced as a result of being treated with NE3107. Patients reported 14 unique change-related concepts related that were categorized as follows: cognitive (2 concepts), psychological (4 concepts), behavioral (2 concepts), physical (5 concepts), and no change (1 concept) (Table 3).

The majority of patients reported at least one area of improvement (n=9; 81.8%); two (15.4) experienced no change. Of those who improved, all experienced at least one cognitive improvement (n=9; 100.0%) and a majority reported at least one positive physical or behavioral change (both n=6; 54.5%). Patients who improved experienced an average of 4.7 (SD=1.86, range 1-7) positive changes.

The most commonly reported changes were cognitive and psychological in nature: improvement in memory (n=8; 72.7%) and mood (n=7; 63.6%).

### Memory

“Well, I think my memory—I mean my thinking was sharper, my memory was a little, a little more helpful to me. Perhaps—you know, perhaps I wasn't in the need of writing so many notes to myself or sending message to my cellphone or my telephone. It just—it just had a profound—it made—it just made a big difference in the short-term memory issues.” [N-23-22]

“It may be I can remember things a little faster, and that's potentially the benefit that I'm getting.” [N-20-22]

“I mean just not having those brain freezes was a very big thing for me, I just felt more normal again . . . ” [N-03-22]

### Mood

“Well, less frustrated, less depressed and really that maybe we can accomplish something with all these new advances.” [N-03-22]

“I think before the study I had a hard time dealing with issues that weren't in the norm, especially at work. If things weren't going right, or I didn't agree with what they were doing at that time I would get very frustrated. I had a lack of tolerance . . . except that now I find myself during the trial, clinical trial, I felt like I wasn't as critical of things that people were doing and I was more accepting. I think that the—and I can't be sure, but I think the medication really kind of leveled me out a little so I didn't hit the high spikes.” [N-18-22]

Over half (n=6; 54.5%) also reported clearer thinking (cognitive) and found themselves to be more engaged (behavioral).

### Clarity

“I felt like I had . . . less pressure on me, so I was lighter, because the thoughts came out more easily and the recognition came out more easily. And then brighter meant, oh, my god, I do have some brain that's working, and I love it.” [N-16-22]

“It was like there was clouds, and then the clouds started to slowly go away, and then they just evaporated. So, then my brain, for my sake, felt clearer, there was no gargling up in my brain, it's like I knew where I was, what I was doing.” [N-22-22]

### Engagement

“I was more involved in the conversations. You know, sometimes people will be talking, you think that they're—you're listening but you're not. But I really started listening, which is something that was different.” [N-18-22]

“You know, I didn't feel like sitting in the bed all the day so I'd get up and do something and move around and then when I would go to do the dishes which a lot of times I would just leave for the housekeeper to do, I would bring the Google in there and then put on some music and then start dancing while I was doing the dishes [laughs] or doing something like that, you know. So I thought geez, every bit. You know, every bit could help with something like this. So yeah, I was doing stuff like that. Yeah, I think I was doing a lot of things that I hadn't done before.” [N-17-22]

Four patients (36.4%) reported feeling hopeful (psychological) more energetic (physical).

### Feelings of Hope

“It helped me feel more secure, not as fearful, same thing. Like I say, hopeful, which is such a big deal and maybe a very good investment, [laughs] but that's beside the point, because without your health you don't have anything. The hope thing weighs more than anything, I mean that's life, you know, where there's hope there's life, so I'm not sure what else to say—where would I be without my mind?” [N-03-22]

### Energy

“I was able to take out an afternoon nap, I was able to get up earlier, I still go to bed a little earlier, but I feel that's because I'm off the meds and I drastically, really, want

| | | | | | | | | | | | | |
|---|---|---|---|---|---|---|---|---|---|---|---|---|
| ID | 03 | 16 | 07 | 18 | 10 | 02 | 14 | 17 | 20 | 22 | 23 | Total |
| | | | | | | | | | | | | (n %) |

them back. I go out and do more things, I'm more active, I walk more, I do more, it's fun for me, I think, because I'm getting sleep.” [N-16-22]

Other less frequently reported changes included fewer headaches and improved self-care (both n=2; 18.2%), and improvements in sleep, movement, weight loss, independence, and overall improvement (all n=1; 9.1%).

### Headaches

“I have dealt with a headache issue for—for a long time and, and interestingly enough I found some remission of the headache issue when I started treatment with Dr. [Name]. That was a blessing. A big blessing.” [N-23-22]

### Self-Care

“I didn't take as good of care of myself as I should have. I didn't—I wasn't exercising regularly. Yeah, no, they are different. There are things that I—when I realized that I was dealing with this and that I was taking the medication, I realized that I had—I had to in general you know, pick up my game of taking care of myself and my aging—my aging brain.” [N-23-22]

Saturation of these concepts was reached within the first 10 interviews, after which no new concepts emerged (Table 3).

TABLE 3
Changes Due to 17α-ethynylandrost-5-ene-3β,7β,17β-triol Treatment (Patients; N = 11)

| Cognitive | | | | | | | | | | | |
|---|---|---|---|---|---|---|---|---|---|---|---|
| Difficulty with | S | P | P | S | S | S | S | | S | 8 | (72.7) |
| Memory | | | | | | | | | | | |
| Difficult with | S | S | P | S | S | | | S | | 6 | (54.5) |
| Clarity | | | | | | | | | | | |
| Psychological | | | | | | | | | | | |
| Improvement | S | S | | S | S | P | | P | S | 7 | (63.6) |
| in Mood | | | | | | | | | | | |
| Feelings | S | S | S | | | | | | S | 4 | (36.3) |
| of Hope | | | | | | | | | | | |
| Feelings of | | | | | S | | | | | 1 | (9.1) |
| Independence | | | | | | | | | | | |
| General | | | | | | | | S | | 1 | (9.1) |
| Improvement | | | | | | | | | | | |
| Behavioral | | | | | | | | | | | |
| Improvement | S | S | | S | S | S | | | S | 6 | (54.5) |
| in Engagement | | | | | | | | | | | |
| Improvement | | | | | | S | | | S | 2 | (18.2) |
| in Self Care | | | | | | | | | | | |
| Physical | | | | | | | | | | | |
| Improvement | | P | | P | S | S | | | | 4 | (36.3) |
| in Energy | | | | | | | | | | | |
| Improvement | | | | | | | | S | S | 2 | (18.2) |
| in Headaches | | | | | | | | | | | |
| Improvement | | P | | | | | | | | 1 | (9.1) |
| in Sleep | | | | | | | | | | | |
| Improvement | | | | S | | | | | | 1 | (9.1) |
| in Movement | | | | | | | | | | | |
| Weight Loss | | | | | S | | | | | 1 | (9.1) |

| | | | | | | | | | | | | |
|---|---|---|---|---|---|---|---|---|---|---|---|---|
| No Change | | | | | P | | P | | | | | 2 |
| # of New | 5 | 2 | 0 | 1 | 1 | 2 | 0 | 1 | 0 | 2 | 0 | 14 |
| Concepts | | | | | | | | | | | | |
| % of New | 35.7 | 14.3 | 0.0 | 7.1 | 7.1 | 14.3 | 0.0 | 7.1 | 0.0 | 14.3 | 0.0 | |
| Concepts | | | | | | | | | | | | |
| Cumulative % | 35.7 | 50.0 | 50.0 | 57.1 | 64.3 | 78.6 | 78.6 | 85.7 | 100.0^{a} | 100.0 | 100.0 | |
| of New Concepts | | | | | | | | | | | | |
| Total Concepts | 5 | 7 | 3 | 6 | 1 | 7 | 1 | 5 | 1 | 4 | 6 | |
| per Interview | | | | | | | | | | | | |

S = Spontaneous;
P = Probed;
Bold = first appearance of concept
^{a}Saturation achieved at 10^{th} interview

All of the patients who experienced a positive change (n=9; 100.0%) felt that at least one was meaningful; for most (n=7; 77.8%), it was improvement in memory (Table 4).

“You know, . . . those are the most valuable things for your day-to-day interactions with people and so . . . I think what tends to isolate people who find their having problems with it is that they're not sure what to say, they don't want to say, you know, you don't want to say the wrong thing, you want to make sure that you remember this perfectly, otherwise, people are going to correct you and that's always not good.” [N-23-22]

“I used to write everybody's name down so I wouldn't forget their names. I don't think I do that now and if I do then I ask [Study Partner].” [N-18-22]

Over half also found improvements in clarity and engagement to be meaningful or important to them (both n=5; 55.6%).

### Clarity

“Well, I enjoy books and it showed I was able to concentrate on them more and . . . then I was also able to concentrate on other creative things more. So I think it all starts kind of with that . . . ” [N-02-22]

### Engagement

“It helps in a conversation. [laughs] It helps to be confident that you can finish a sentence if you want to tell somebody something, that you have all the words there without having to practice. I would probably be more talkative if I felt more confident about remembering the dates and the time or the names and the locations.” [N-20-22]

TABLE 4
Meaningful Changes Due to Treatment
(Patients Reporting Improvement; n = 9)

| Meaningful Change | Total n (%)¹ |
|---|---|
| Memory | 7 (77.8) |
| Clarity | 5 (55.6) |
| Engagement | 5 (55.6) |
| Mood | 5 (55.6) |
| Energy | 4 (44.4) |
| Headaches | 3 (33.3) |
| Feelings of Hope | 2 (22.2) |
| Movement | 1 (11.1) |
| Weight Loss | 1 (11.1) |

¹Not mutually exclusive

Three patients (27.3%) described worsening after stopping NE3107. They noted a return to pretrial symptoms, including frustration, decreased energy levels, and a general sense of “heading toward before the study” since they are “not getting any better now.” [N-16-22].

“ . . . because then when I stopped it—I could tell it's just been harder [to lose weight]. So and I don't know if that's true or not, if it's the drugs. But—and maybe again, that's because then I was concentrating more on what I could eat and energy and moving more.” [N-02-22]

At the end of their interview, patients were asked to reflect on their use of NE3107 during the trial, including their ability to comply with the medication regimen.

All patients (n=11; 100.0%) felt that the study medication was easy to take.

“Completely the easiest thing to take, no problems at all.” [N-22-22]

“It was easy because it was a capsule, if that's what you mean and it went down easy . . . ” [N-02-22]

Most (n=10; 90.9%) had no issues taking the study medication at the frequency directed (for example, twice a day) and that it was easy to remember to take (n=8; 72.7%).

“It was no problem because I have them with my pills in containers and you know, morning, evening or morning, afternoon, night. And they're done at—like a week in advance.” [N-17-22]

“For me it was really easy. I had a little chart. I'm very organized, anyway, I'm a little bit anal that way. I took it in the morning, I checked it off I took it in the evening, I checked it off He didn't ask me to save the blister packs, so I threw them away, but I had a copy of—I kept the chart so I was always able to show him that . . . I never missed any, and I was very good about taking them, actually.” [N-20-22]

Three patients (27.3%) felt it took extra effort to remember to take the study medication, at least at the start of the trial.

“Well, at first it was horrid, but I read everything and I put it where my marker place was so that I would see it and know to do it, but I had that plan already decided, but the first week I did miss some, and then I went, oh, this is helping me, I'm going to take it forever.” [N-16-22]

“Well, I—yeah, I try hard to remember to take it. If it's on like a card so to speak and I try really hard to remember to take it in terms of the timetable that I was supposed to take it within.” [N-07-22]

Seven study partners completed interviews alongside patients (63.6%). Following the patient's interview, study partners were asked to separately offer their own perspectives on the symptoms and impacts of the patient's degenerative dementia and any changes they noticed as a result of the trial.

At the beginning of the interview, study partners were asked to describe difficulties they noticed in their partner's memory or thinking prior to the NE3107 trial. Study partners described 9 concepts regarding patients' psychological (1 concept), cognitive (2 concepts), behavioral (2 concepts), and physical (2 concepts) issues, as well as impacts on everyday life (2 concepts) (Table 5).

Study partners relayed an average of 4 (SD=1.53, range 2-6) problems and impacts related to the patients' degenerative dementia.

Patients' cognitive problems were mentioned by all study partners (n=7; 100.0%), including issues with clarity (n=6; 85.7%) and memory (n=5; 71.4%).

### Clarity

“Yeah, a few times before the study where it had been somewhere she had gone a million times and she was like, wait, I got confused, I think I turned the wrong way. Usually, she uses google maps for stuff, but for, obviously, places she knows how to get to she wouldn't, that's sometimes when she ran into trouble.” [N-16-22]

“He said I sent her out on an errand, she left 10:00 in the morning, we couldn't find her, he said we didn't find her until 5:00 in the afternoon. She wandered for nine miles, she was clueless as to where she was, she has no idea. We finally got her on the phone, we told her to tell us where she was, she said I'm such and such.” [N-22-22]

### Memory

“Well, she couldn't remember words, she couldn't remember conversations or things that had happened, especially recently, short-term was worse than longterm, and then she couldn't remember certain processes, even things that she'd done on a regular basis for a long time.” [N-16-22]

“ . . . there are many, many instances in which she will ask a question, and half an hour later she'll ask the same question, and half an hour later she'll ask the same question.” [N-14-22]

All study partners described issues with patients' mood (psychological; n=7; 100.0%).

“ . . . he would at times get very short-tempered and angry. I imagine that's again, not wanting to be dependent . . . ” [N-07-22]

“She was very uncooperative in that office, she wouldn't do eye-to-eye contact with him, she wouldn't answer his questions, she was a real smart-ass with him. She was also that way with the dentist, just real abrasive attitude . . . ” [N-22-22]

Less commonly, study partners described patients requiring dependence on others (n=3; 42.9%), and difficulties with engagement and self-care (both n=2; 28.6%).

### Dependence on Others

“I'm waiting on her hand and foot, I'm feeding her, her weight went from 100, now I've got her at almost 140, she's gone from a size extra small back to her normal, which is a medium.” [N-22-22]

“I mean I had to drive whether I felt like it or not because he couldn't drive. He couldn't I'd have to repeat over and over again.” [N-07-22]

### Engagement

“I also noticed he used to be a great artist. Not a great artist. Let me put it this way, he could draw anything. You know, if I gave him something and I said draw me this so I can use it on as a card, he could do that. And noticed before the study that all of a sudden that skill wasn't there anymore.” [N-18-22]

### Self-Care

“ . . . she was unrecognizable to . . . my granddaughter and to myself and to my husband. Her hair was out to here, dry, her skin was, withdrawn, she looked like she'd aged 20 years. [Patient Name] has been extremely well-groomed, extremely. Her breath was bad, her skin was bad, her clothes hung on her, she was an extra small, the clothes that she had on fit somebody that was a medium, so they just hung on her.” [N-22-22]

Less commonly, study partners described patients' difficulty with energy, movement, and impacts at work (all n=1; 14.3%).

TABLE 5
Pre-Trial Symptoms and Impacts (Study Partners; N = 7)

| | | | | | | | | Total | |
|---|---|---|---|---|---|---|---|---|---|
| ID | 16 | 07 | 18 | 14 | 17 | 22 | 23 | (n %) | |
| Psychological | | | | | | | | | |
| Difficulty with | X | X | X | X | X | X | X | 7 | (100.0) |
| Mood | | | | | | | | | |
| Cognitive | | | | | | | | | |
| Difficulty with | X | X | | X | | X | X | 5 | (71.4) |
| Memory | | | | | | | | | |
| Difficulty with | X | X | X | X | X | X | | 6 | (85.7) |
| Clarity | | | | | | | | | |
| Impacts | | | | | | | | | |
| Dependence | | X | | X | | X | | 3 | (42.9) |
| on Others | | | | | | | | | |
| Difficulty with | X | | | | | | | 1 | (14.3) |
| Clarity | | | | | | | | | |
| Behavioral | | | | | | | | | |
| Difficulty with | X | | X | | | | | 2 | (28.6) |
| Engagement | | | | | | | | | |
| Difficulty with | | X | | | | X | | 2 | (28.6) |
| Self-Care | | | | | | | | | |
| Physical | | | | | | | | | |
| Difficulty with | X | | | | | | | 1 | (14.3) |
| Energy | | | | | | | | | |
| Difficulty with | | | X | | | | | 1 | (14.3) |
| Movement | | | | | | | | | |

| | | | | | | | | |
|---|---|---|---|---|---|---|---|---|
| # of New Concepts | 6 | 2 | 1 | 0 | 0 | 0 | 0 | 9 |
| % of New Concepts | 66.7 | 22.2 | 11.1 | 0.0 | 0.0 | 0.0 | 0.0 | |
| Cumulative % of | 66.7 | 88.9 | 100.0 | 100.0 | 100.0 | 100.0 | 100.0 | |
| New Concepts | | | | | | | | |
| Total Concepts | 6 | 5 | 4 | 4 | 2 | 5 | 2 | |
| per Interview | | | | | | | | |

Bold = first appearance of concept

When asked what symptoms or impacts were the most challenging before the trial, the majority of study partners brought up the patient's mood (n=4; 57.1%).

“Like she just was always picking fights with me and I think it was she was upset about always losing stuff and never getting things done. So it was just like she was just so unpleasant a lot of the times and I noticed it very definitely. So that was definitely the most difficult.” [N-17-22]

Three study partners described issues with the patient's clarity as most challenging (42.0%).

“I guess just seeing that his thinking was deteriorating and that he wasn't as sharp as he had always been, and asking me for more help along certain lines that he never did before, getting things done, making sure I'm there all the time to help him out . . . ” [N-18-22]

Less commonly, study partners mentioned difficulties with memory (n=2; 28.6%), energy, and dependence on others (both n=1; 14.3%) as challenging.

Study partners described 11 unique concepts related to changes patient's experienced as a result of the trial, including “no change”. All concepts mentioned mirrored those described by patients; no new concepts emerged. Themes of the changes mentioned were cognitive (2 concepts), psychological (2 concepts), behavioral (3 concepts), physical (3 concepts), and no change (1 concept). Six of the study partners (85.7%) described improvements as a result of the trial, in accordance with their patient partners, and one (14.3%) agreed with the patient that no changes were experienced (N-14-22) (Table 6).

The most commonly mentioned changes were psychological and cognitive: improvements in mood (n=6; 85.7%) and memory (n=5; 71.4%).

### Mood

“Well, it was a relief because she was feeling better, she was, obviously, more relaxed, which was nice, and smiling more, which [laughs] was nice, and laughing more, which was great. She didn't need as much help, and a little bit less tension [laughs] between her and dad, so that was an easing.” [N-16-22]

“She was so much more pleasant. She stopped fighting with me all the time.” [N-17-22]

### Memory

“ . . . she definitely started remembering things more and being able to function on her own more.” [N-16-22]

“ . . . two people who spoke with him on the phone or happened to see him commented that they could see a change or and hear a change in his hesitance in responding and trying to think of what he wanted to say. I mean he's still doing it at this point, but not as much as he was before.” [N-07-22]

Improvements in clarity (cognitive), engagement (cognitive), and independence (behavioral; all n=4; 57.1%) were also reported by study partners.

### Clarity

“So, she didn't have as many problems with like the Excel sheets that she uses and trying to input formulas. She didn't have to maybe have someone drive with her any more, she could do grocery shopping on her own again, things like that.” [N-16-22]

### Engagement

“ . . . let's say we went to a family event. He would talk to everybody. He would talk to everybody. He walks around and talks to everybody. And then after he's decided he's had enough he'll say to me okay, I'm ready to go home—his whole life . . . But with—during the study he didn't do that. He was really engaged with the people . . . he was okay. He would stay the whole night even sometimes. I mean we've—before that we would—sometimes he would drive separately because I wanted to stay and he didn't want to stay, that kind of thing. But during the study he seemed to let go of that need to be in control of when we go, when we'd leave.” [N-18-22]

### Independence

“She gets up in the morning, she fixes her own breakfast, I make her protein drink, we are now back to being that mother daughter fun person and I'm not so overwhelmed with her care.” [N-22-22]

Three study partners (42.9%) described improvements in the patient's self-care (behavioral) and two (28.6%) mentioned a sense a hope (psychological) following the trial.

### Self-Care

“There was a change in her attitude, and she had more self-care, more self pride, she was participating in her care, and she was functioning better.” [N-22-22]

### Feelings of Hope

“But then after a while it began to—you could—I could see it and I could see that he was looking a little bit better, was presenting himself better. And then came up with some hopefulness, feeling hopeful.” [N-07-22]

Physical changes reported included a decrease in headache frequency and severity (n=2; 28.6%) and improvements in energy (n=2; 28.6%) and overall movement (n=1; 14.3%).

### Headaches

“I think she hardly had any headaches at all for the next three months . . . ” [N-23-22]

### Energy

“Because when she wasn't as tired, she was more herself she was more of a person, so it was more like interacting with mom.” [N-16-22]

### Movement

“He's not shuffling. He's picking up his feet. He's still doing that pretty well.” [N-18-22]

TABLE 6
Changes Due to 17α-ethynylandrost-5-ene-3β,7β,17β-triol
Treatment (Study Partners; N = 7)

| | | | | | | | | Total |
|---|---|---|---|---|---|---|---|---|
| ID | 16 | 07 | 18 | 14 | 17 | 22 | 23 | (n %) |
| Psychological | | | | | | | | |
| Improvement | X | X | X | | X | X | X | 6 (85.7) |
| with Mood | | | | | | | | |
| Feelings of | X | X | | | X | X | | 4 (57.1) |
| Independence | | | | | | | | |
| Feelings | | X | | | | | X | 2 (28.6) |
| of Hope | | | | | | | | |
| Cognitive | | | | | | | | |
| Improvement | X | X | | | X | X | X | 5 (71.4) |
| with Memory | | | | | | | | |
| Improvement | X | X | | | X | X | | 4 (57.1) |
| with Clarity | | | | | | | | |
| Behavioral | | | | | | | | |
| Improvement | X | | X | | X | X | | 4 (57.1) |
| with Engagement | | | | | | | | |
| Improvement | | X | | | X | X | | 3 (42.9) |
| with Self-Care | | | | | | | | |
| Physical | | | | | | | | |
| Improvement | X | | | | | X | | 2 (28.6) |
| with Energy | | | | | | | | |
| Improvement | | | | | | X | X | 2 (28.6) |
| in Headaches | | | | | | | | |
| Improvement | | | X | | | | | 1 (14.3) |
| in Movement | | | | | | | | |
| No Change | | | | X | | | | 1 (14.3) |
| # of New Concepts | 6 | 2 | 1 | 1 | 0 | 1 | 0 | 11 |
| % of New Concepts | 54.5 | 18.2 | 9.1 | 9.1 | 0.0 | 9.1 | 0.0 | |
| Cumulative % of | 54.5 | 72.7 | 81.8 | 90.9 | 90.9 | 100.0^{a} | 100.0 | |
| New Concepts | | | | | | | | |
| Total Concepts | 6 | 6 | 3 | 1 | 6 | 8 | 4 | |
| per Interview | | | | | | | | |

Bold = first appearance of concept
^{a}Saturation achieved at 6^{th} interview with study partner

Each of the 10 concepts related to improvement was considered meaningful to at least one study partner (Table 7). Among the 6 study partners who described improvements in the patients, all (100.00%) felt that the improvements in the patients' mood were meaningful.

“It was just so nice to not see her so frustrated and down. And, yeah, she's a happy person, and she's been a happy person all my life, and then she wasn't, and it was weird and sad. It was like oh, okay, there you are again.” [N-16-22]

“I can't tolerate unkind people and I can't tolerate rudeness, and I can't tolerate people getting nasty, and she was doing that. So, now, after she got on that drug, she's back, her personality is back, she's pretty again.” [N-22-22]

Study partners also felt that the cognitive improvements reported in memory and clarity (both n=3; 50.0%) were meaningful.

### Memory

“I mean it made me happy to see her doing something, like I don't know whether the Post-It notes helped with recall or the drug itself, but it was it made me happy that she was implementing tools to help her be successful, because I think that expecting things to just get better without having aides is not reasonable.” [N-17-22]

### Clarity

“Yeah, it was a real estate book and it was something that he used to live and breathe by until all of this occurred. So it was good to see it again. I knew that was a step forward, an important step forward.” [N-07-22]

TABLE 7
Meaningful Changes Due to Treatment
(Study Partners Reporting Improvement; n = 6)

| Meaningful Change | Total n (%)¹ |
|---|---|
| Mood | 6 (100.0) |
| Memory | 3 (50.0) |
| Clarity | 3 (50.0) |
| Engagement | 2 (33.3) |
| Feelings of Independence | 2 (33.3) |
| Energy | 1 (16.7) |
| Headaches | 1 (16.7) |
| Feelings of Hope | 1 (16.7) |
| Movement | 1 (16.7) |
| Self-Care | 1 (16.7) |

¹Not mutually exclusive

Study partners were asked if they would recommend NE3107 to others. The vast majority (n=5; 71.4%) reported that they definitely would, and one (14.3%) offered a cautiously positive recommendation.

“Well, depending upon their symptoms, I would definitely say it was worth a try. He didn't seem to have any negative side effects or anything from taking it. So I think it was well worth it to do it. And so I would recommend it based on that.” [N-18-22]

“ . . . I think I would definitely recommend it to people who have any visible changes and I'm sure it would be helpful to people with more significant changes as well, but I don't know if I would recommend it to people who are like far gone because I don't know that that's what it's designed for.” [N-17-22]

One study partner (14.3%) would not recommend the study drug, as neither they nor the patient noted any significant changes as a result of treatment.

“Well, not at all, because I didn't see any result. I couldn't . . . I mean, [Name] kept saying some of his patients are having very serious improvement because of this. I'd recommend people look at something like this if the publicity says this helps 6/10 people, fine, but it didn't help [Patient Name] that I could see.” [N-14-22]

At the end of their interview, study partners were asked for any final thoughts about their experience living with and/or caring for someone with memory or thinking problems. Most (n=5; 71.4%) described negative emotional and physical effects on themselves and other people in the patients' lives.

“I became depressed, anxiety, I neglected my self-care, because I was so focused on her. I knew I had to get her better and I knew I had to do it quick, because whatever was I didn't want it to progress.” [N-22-22]

“Just that it can be very difficult for everybody, and that if there is anything that can help that's amazing, we need more of that.” [N-16-22]

Two study partners (28.6%) described the benefits of having a support system.

“It's not a great experience, but it's something you can live through, and we've been lucky that we have lots of friends who are particularly friends of [Patient Name]'s who are helping, and our family is great. So we're lucky compared to 99 100 people.” [N-14-22]

“It takes a village, but you cannot do this without help, and her business partner helped, her clients helped, her boyfriend helped, my family helped, my doctor helped, social services helped, but you've got to reach out and request that help, I had no fear of asking.” [N-22-22]

Finally, two study partners mentioned a desire to have the patient continue taking NE3107.

“So I mean if there was an opportunity to take the meds longer, I would definitely like to see whether it continued in the same vein even though he wasn't in the study type of thing. It would be very interesting for me to see.” [N-18-22]

“ . . . I'm very grateful that we've somehow been apart of this new discovery on this and I'm very optimistic about it and I, you know, I think that this is—I just want to make sure that on a personal basis that we have access to it.” [N-23-22]

The interview results were compared with changes from baseline to Month 3 in cognitive functioning as measured by the ADAS-Cog11, and the patient-completed Global Rating of Change (GRC) at Month 3 in the Phase 2 trial (Table 8).

The ADAS-Cog 11 is a performance-based assessment of cognitive function that includes 11 patient-completed tasks that measure word recall, objective/figure naming, command following, constructional praxis, ideational praxis, orientation, word recognition, test direction recall, spoken language, comprehension, and word-finding difficulty. Scores range from 0-70 where higher scores indicate more severe cognitive impairment (i.e., decreases in score over time indicate improvement in cognition). The GRC is a single item that asks patients to rate changes in their condition, abilities, and overall sense of well-being from the start of the trial. Scores range from −5 (“Very much worse”) to 0 (“Unchanged”) to +5 (“Very much better”).

Of the 23 patients in the Phase 2 trial, the average patient had improvement in cognitive function; just over half (n=13; 56.5%) had a decrease in ADAS-Cog 11 scores from baseline to Month 3. A majority (n=18; 78.3%) reported improvement in their condition as measured by the GRC at Month 3.

Similarly, the majority of the interviewed patients had an improvement in cognitive function as measured by the ADAS-Cog 11 (n=7; 63.6%), reported an improvement in their condition on the GRC at Month 3 (n=9; 81.8%). These findings correspond to most participants (n=9; 81.8%) describing improvements in memory or clarity during the interview. Of note, the GRC score and cognitive change reported during the interview (i.e., change in memory or clarity) were not always congruent with the direction of the score change of the ADAS-Cog 11 (Table 8). The majority of patients (n=7; 63.6%) were either congruent across all three assessments (i.e., improvement across ADAS-Cog 11, GRC, and Exit Interview) or congruent on the trial assessments (i.e., improvement on ADAS-Cog-11 and GRC) but not the interview results (i.e., no change reported in memory or clarity during the interview).

TABLE 8
Comparison of ADAS-Cog 11, GRC, and Exit Interview

| | | | | Patient | | |
|---|---|---|---|---|---|---|
| | | | | Global | | |
| | | | | Rating of | Cognitive | Comparison |
| | ADAS-Cog-11 | | | Change | Change from | (ADAS-Cog 11/ |
| Subject | Baseline | 3M | Change¹ | (GRC)² | Interview³ | GRC/Interview)⁴ |
| N-16-22 | 21.00 | 11.00 | −10.00 | 4 | Improvement | +/+/+ |
| N-14-22 | 14.00 | 9.00 | −5.00 | 2 | No Change | +/+/NC |
| N-03-22 | 11.33 | 6.00 | −5.33 | 3.5 | Improvement | +/+/+ |
| N-07-22 | 19.33 | 14.67 | −4.67 | 1 | Improvement | +/+/+ |
| N-10-22 | 5.00 | 2.33 | −2.67 | −1 | No Change | +/−/NC |
| N-18-22 | 10.00 | 8.67 | −1.33 | 4 | Improvement | +/+/+ |
| N-20-22 | 4.00 | 3.33 | −0.67 | 3 | Improvement | +/+/+ |
| N-02-22 | 5.00 | 5.67 | +0.67 | −5 | Improvement | −/−/+ |
| N-23-22 | 9.33 | 10.00 | +0.67 | 3 | Improvement | −/+/+ |
| N-22-22 | 27.67 | 29.00 | +1.33 | 5 | Improvement | −/+/+ |
| N-17-22 | 6.67 | 9.33 | +2.67 | 4 | Improvement | −/+/+ |

¹Any decrease in score on the ADAS-Cog 11 indicates an improvement. A reduction of 3.1 to 3.8 points has been found to be a clinically significant worsening on the ADAS-Cog (Schrag and Schott 2012)
²Scores on the Patient GRC range from −5 (Very Much Worse) to 0 (Unchanged) to 5 (Very Much Better)
³Patient description of change in “Memory” or “Clarity” during exit interview
⁴+ = Improvement; NC = No change; − = Worsening

The goal of these exit interviews was to qualitatively characterize the patient experience with NE3107, an anti-inflammatory insulin sensitizing agent, during an open-label Phase 2 trial. To evaluate these objectives, approximately half (n=11; 47.8%) of the 23 patients who completed NE3107-TRP-001 participated in semi-structured exit interviews led by experienced and trained interviewers over videoconference. Each interview was divided into four parts: 1) elicitation of experience with degenerative dementia, 2) changes due to treatment and meaningfulness of those changes, 3) experience with NE3107 during the clinical trial, and if applicable 4) study partner perceptions of the patient's changes during the trial and their trial experience.

Interviewed patients reported 14 unique concepts related to changes experienced during NE3107-TRP-001. Saturation of concepts was reached within the first ten interviews. These patients reported improvements in cognition (memory, clarity) consistent with the goals of treatments for cognitive decline. This finding is consistent with the changes in cognitive functioning measured by the ADAS-Cog 11 observed in the interviewed patients. All interviewed patients had either a decrease (indicating improvement) or increase in their ADASCog 11 score that was less than the score change (3.1 to 3.8 points) indicative of clinically significant worsening in cognitive functioning over a 6-month period (Schrag and Schott 2012). This finding should be interpreted cautiously given the 3-month follow up period in NE3107-TRP-001.

Additionally, unexpected benefits of treatment with NE3107 were improvements in other aspects of function and well-being, including: psychological (improved mood, feelings of hope, independence), behavioral (self-care, engagement), and physical (weight loss, increased energy, fewer headaches, improved sleep and movement). The improvements experienced during the trial were considered meaningful, especially with regards to cognitive (to patients) and mood (to study partners).

Although qualitative data obtained from interview participants is generally considered to be reliable, it cannot be confirmed as to whether these findings are representative of all patients who completed the NE3107-TRP-001 trial. Less than half of the patients/study partners who completed the trial volunteered to complete the exit interviews, so these findings are limited to those who opted in to provide their feedback. However, two of the interviewed patients did not report any changes as a result of treatment, so the exit interviews were not limited to only those who reported improvement. Additionally, open label designs limit the interpretation of patient reported data because the patients'/study partners' knowledge of the treatment regimen may lead to systematic overestimation or under estimation of treatment benefit.

Exit interviews can be used to gather qualitative descriptions of pre-trial disease burden as well as changes experienced during a trial, including whether and why any improvements were meaningful (US FDA 2022). The qualitative data from these interviews highlight that the changes observed by patients and their study partners included other domains (for example, psychological, behavior, and physical function) in addition to improved cognitive function, as well as activities of daily living. These broader changes were meaningful to patients/study partners and provide preliminary data that can be used to develop a clinical outcome assessment strategy to differentiate NE3107 from competitors. Measurement of the broad spectrum of benefits that may result from treatment with NE3107 will aid in interactions with both regulatory and HTAs and in communicating the benefits of NE3107 from the patient/caregiver perspective. The use of exit interviews helps characterize the treatment benefit of NE3107 by translating the results over time from neuropsychological tests, measures of neurophysiological health, and metabolic/serological analyses into what is a meaningful change in the lives of patients and their caregivers. Eliciting patient/study partner descriptions allows for the opportunity to not only identify health concepts of greatest importance to patients with MCI due to degenerative dementias, but also to uncover unexpected benefits which can be explored in future trials to fully characterize the value of NE3107 to patients living with dementia and their caregivers.

The exit interviews served to fully characterize and understand the patient/study partner experience with NE3107 during NE3107-TRP-001. Outcomes assessments in future trials of NE3107 could include assessments of cognition (memory and thinking), psychological, behavioral, and physical domains, and impacts on daily life. Consideration should also be given to additional exit interviews as part of a blinded trial to complement quantitative assessments by providing a rich description of the patient and caregiver experience, including meaningful changes.

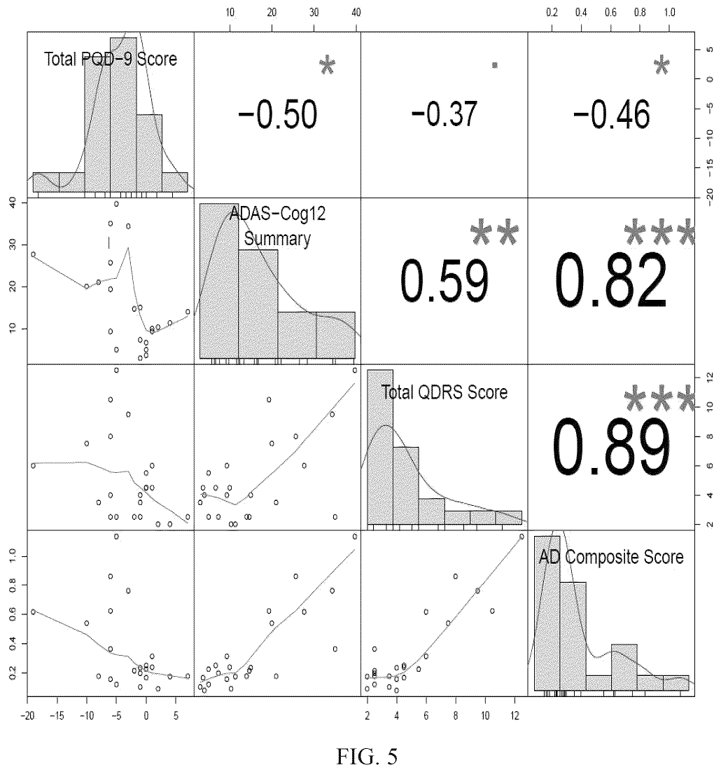

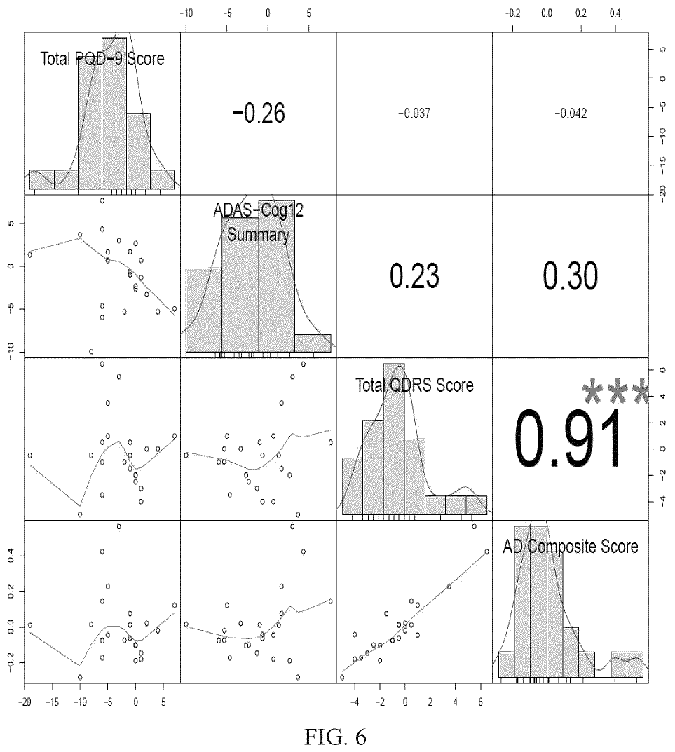

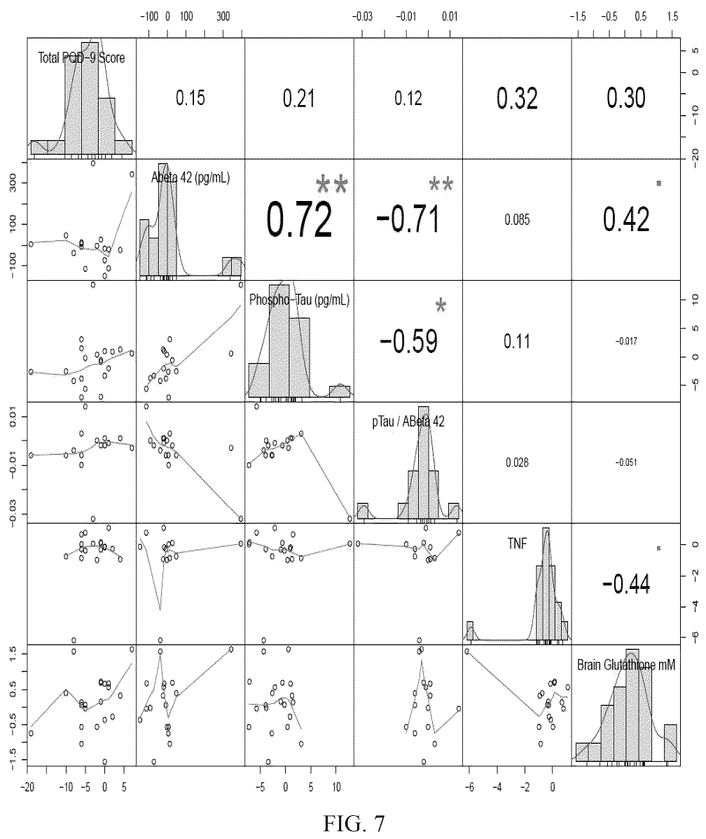

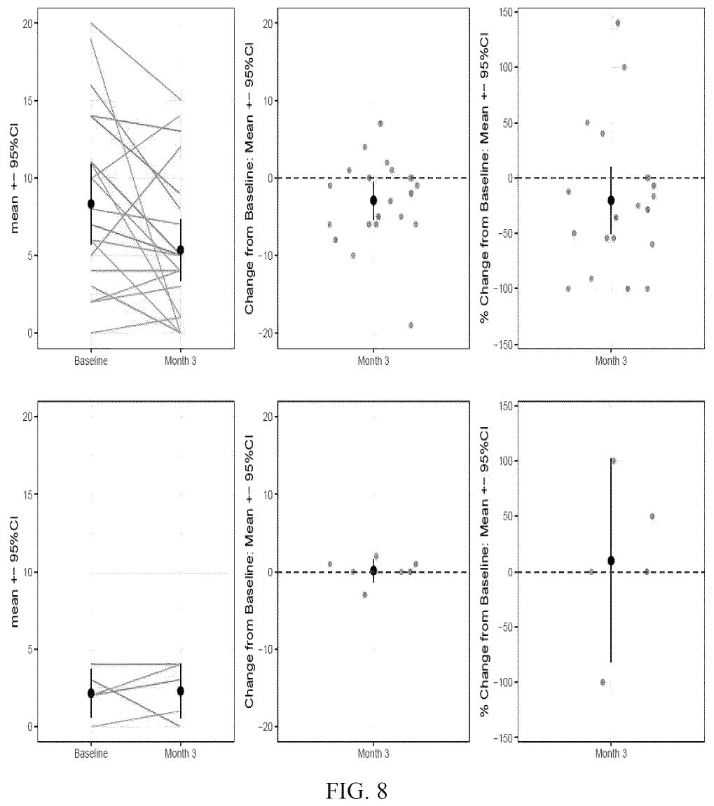

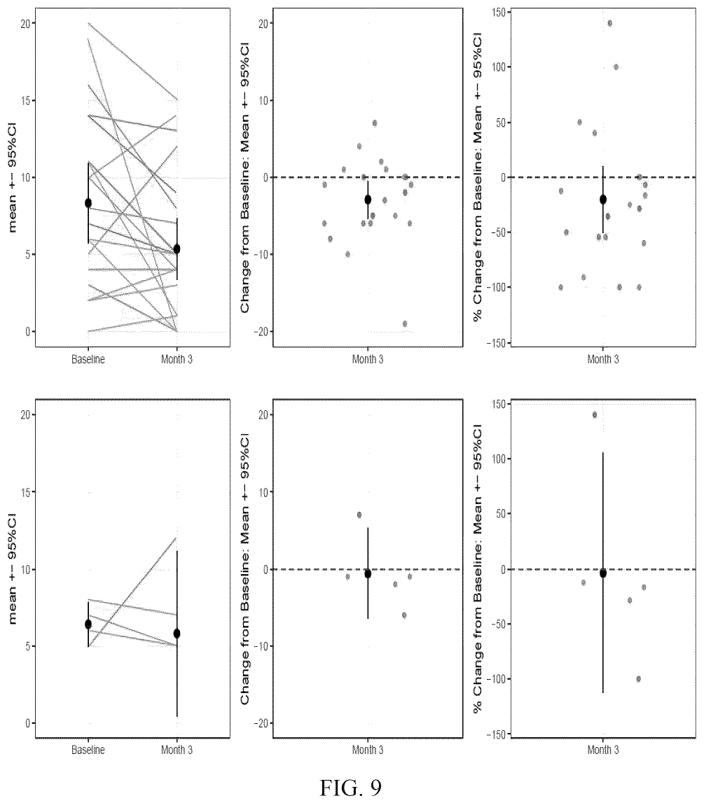

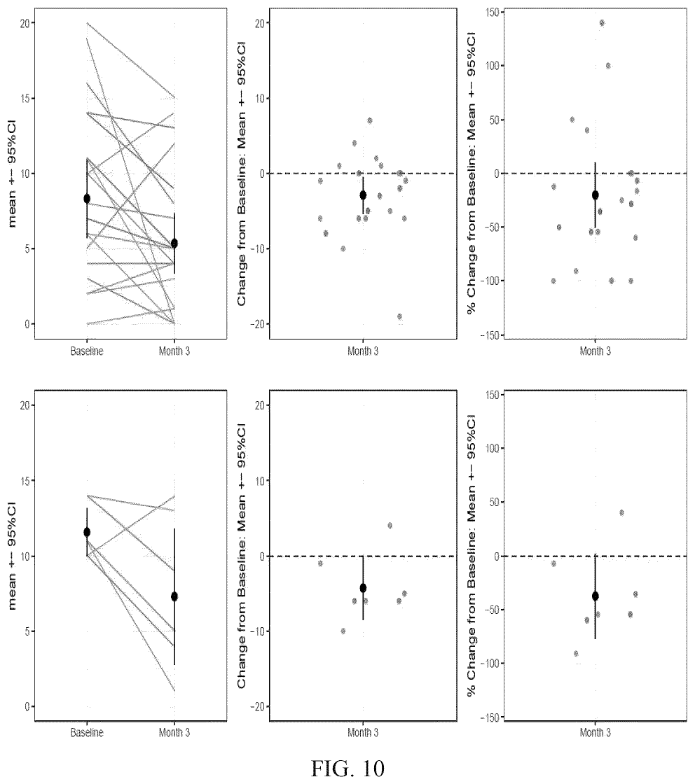

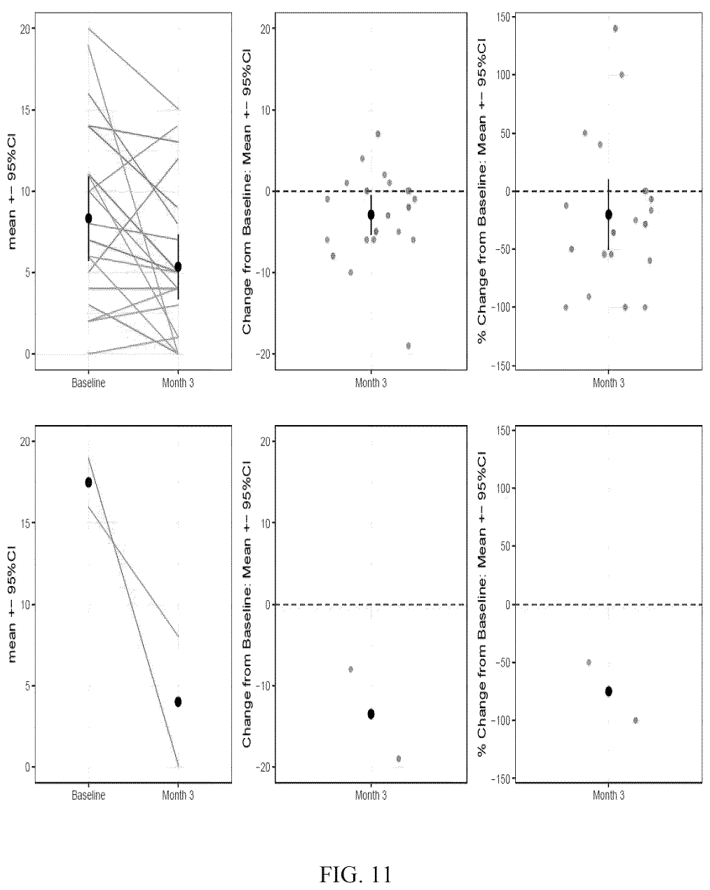

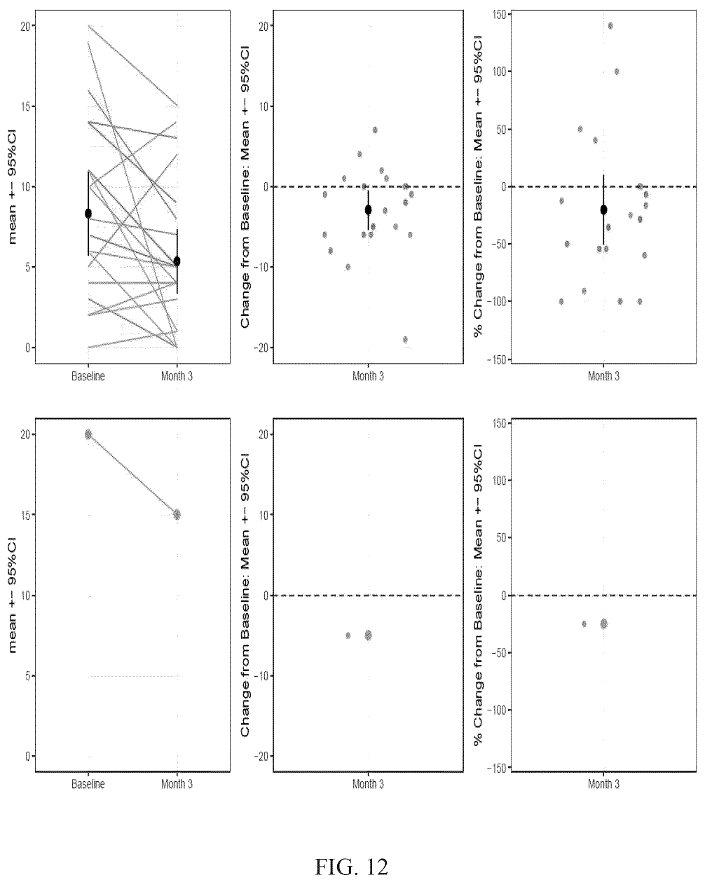

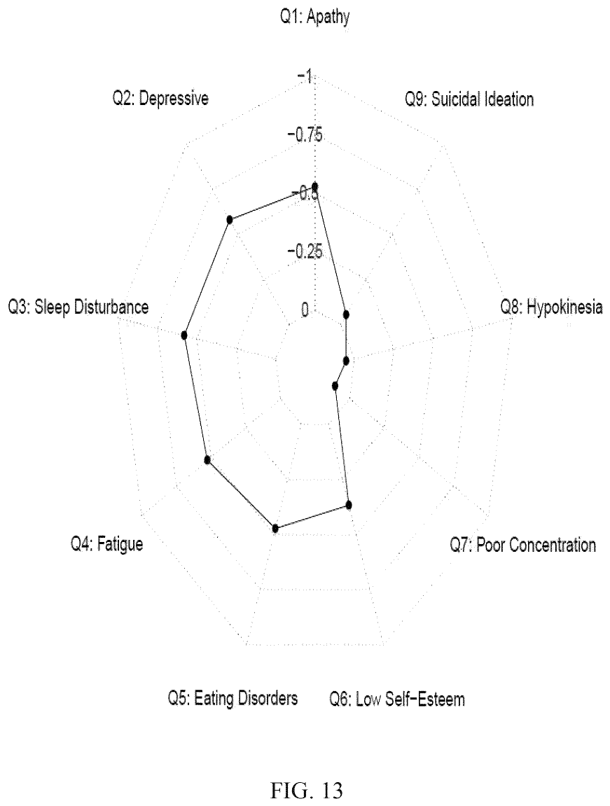

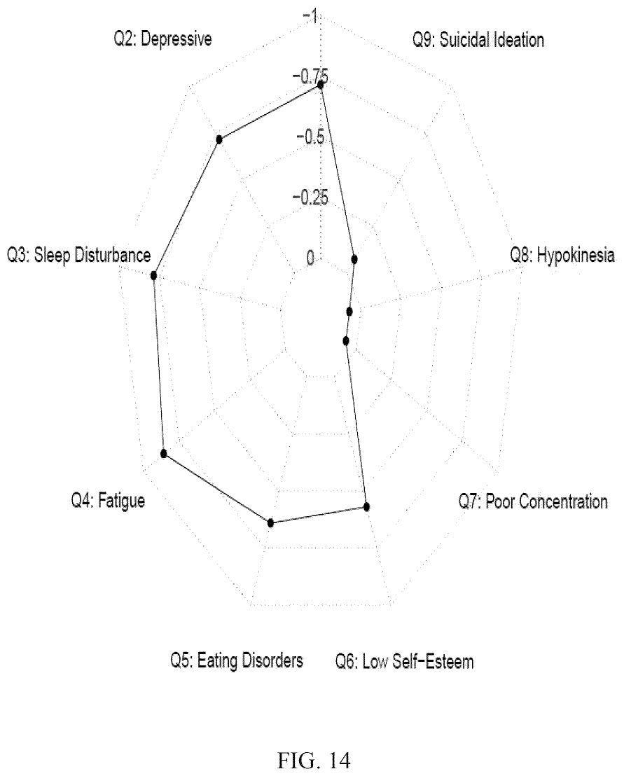

FIGS. 5-7 illustrate the correlation of change from baseline in PHQ-9 and baseline ADAS-Cog12, Total QDRS and AD composite Score, indicating improvement in depression is linked to the patient's baseline cognitive status, which largely influenced by inflammation, and thus may be susceptible to improvement from the anti-inflammatory activity of NE3107. The mean PHQ-9 score decreased by an average of 4.3 points. Patients with the highest level of depression (baseline PHQ-9 score ≥5) experienced the greatest improvement in depression symptoms. FIGS. 8-12 illustrate results of total PQD-9 score of the enrolled subjects. FIG. 13 illustrates a radar plot of PHD-9 of the mean change from baseline for each domain scores with subjects compared to baseline. Patients with mild to severe depression (baseline PHQ-9 score ≥5) had greater improvement, compared to all patients, in several individual PHQ-9 domains evaluating apathy, depressive symptoms, sleep disturbance, fatigue, eating disorders, and low self-esteem. FIG. 14 illustrates a total PQD-9 score regression of all enrolled population.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Although the invention has been described with reference to embodiments and examples, it should be understood that numerous and various modifications can be made without departing from the spirit of the invention. Accordingly, the invention is limited only by the following claims.

What is claimed is:

1. A method to treat, reduce or ameliorate of a neurodegenerative condition or disease in a subject in need thereof, the method comprising:
identifying a subject having a waist to hip ratio greater than or equal to approximately 0.80; and
administering to the subject a therapeutically effective amount of a composition comprising 17α-ethynylandrost-5-ene-3β,7β,17β-triol and at least one pharmaceutically acceptable excipient.

2. The method of claim 1, wherein the subject has a waist to hip ratio greater than approximately 0.90.

3. (canceled)

4. (canceled)

5. The method of claim 1, wherein the neurodegenerative condition is a mild or moderate cognitive impairment.

6. The method of claim 5, wherein the mild or moderate cognitive impairment is late onset Alzheimer's disease or Alzheimer's disease, or probable Alzheimer's disease.

7. The method of claim 1, wherein the neurodegenerative condition or disease is dementia.

8. (canceled)

9. (canceled)

10. The method of claim 1, wherein the subject experiences an improvement in overall cognitive function.

11. The method of claim 10, wherein the improvement in overall cognitive function is from about 5% to about 75%.

12. The method of claim 1, wherein the subject experiences an improvement in memory recall.

13. The method of claim 12, wherein the improvement in memory recall is from about 5% to about 75%.

14. The method of claim 1, wherein the subject experiences an improvement in memory loss.

15. The method of claim 14, wherein the improvement in memory loss is from about 5% to about 75%.

16. The method of claim 1, wherein the subject experiences an improvement in mild cognitive dysfunction.

17. The method of claim 16, wherein the improvement in mild or moderate cognitive dysfunction ranging is from about 5% to about 75%.

18. (canceled)

19. The method of claim 1, wherein the 17α-ethynylandrost-5-ene-3β,7β,17β-triol is administered orally or intravenously.

20. (canceled)

21. The method of claim 1, wherein the subject has a waist to hip ratio greater than or equal to approximately 0.80 and the subject is female; or wherein the subject has a waist to hip ratio greater or equal to approximately 0.90 and the subject is Hispanic or Asian; or wherein the subject has a waist to hip ratio greater or equal to approximately 0.95 and the subject is male other than Asian or Hispanic.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A method to increase intracellular concentration of glutathione in a cerebral cortex, limbic system, cerebellum, or brain stem in a subject in need thereof, the method comprising:
identifying a subject having a waist to hip ratio greater than or equal to approximately 0.80; and
administering to the subject a therapeutically effective amount of a composition comprising 17α-ethynylandrost-5-ene-3β,7β,17β-triol and at least one pharmaceutically acceptable excipient.

34. The method of claim 33, wherein the subject has a waist to hip ratio greater than approximately 0.90.

35. (canceled)

36. (canceled)

37. The method of claim 33, wherein the subject increases the intracellular concentration of glutathione in the cerebral cortex or limbic system by approximately 5% to 100% of their mid-life functional level.

38. The method of claim 33, wherein the subject experiences an improvement in overall cognitive function or memory recall.

39. The method of claim 38, wherein the subject experiences an improvement in overall cognitive function or memory recall ranging from approximately 5% to 100% of their mid-life cognitive function or function before disease onset.

40. (canceled)

41. (canceled)

42. (canceled)

43. (canceled)

44. (canceled)

45. (canceled)

46. (canceled)

47. (canceled)

48. (canceled)

49. (canceled)

50. (canceled)
